(19) United States
(12) Patent Application Publication
(10) Pub. No.: US 2025/0157650 A1
(43) Pub. Date: May 15, 2025

(54) METHODS AND SYSTEMS FOR PREDICTING GENOMIC PROFILING SUCCESS

(71) Applicant: Foundation Medicine, Inc., Boston, MA (US)

(72) Inventors: Richard Sheng Poe HUANG, Cary, NC (US); Douglas A. MATA, Cambridge, MA (US)

(73) Assignee: Foundation Medicine, Inc., Boston, MA (US)

(21) Appl. No.: 18/722,333

(22) PCT Filed: Dec. 6, 2022
(86) PCT No.: PCT/US2022/080996

§ 371 Date: Jun. 20, 2024

Related U.S. Application Data
(60) Provisional application No. 63/292,395, filed on Dec. 21, 2021.

(51) Int. Cl. G16H 50/20; G16H 10/40; G16H 20/10

(52) U.S. Cl. CPC G16H 50/20; G16H 10/40; G16H 20/10

(57) ABSTRACT

Methods for predicting a likelihood of success for performing genomic profiling of a sample derived from a subject are described. In some instances, the method may comprise: receiving data for a plurality of pre-analytical variables associated with the sample; applying the received data to a multivariable model trained to predict outcomes for genomic profiling assays; generating a prediction of the likelihood of success for performing genomic profiling of the sample based on the applied multivariable model; and reporting the prediction of the likelihood of success for performing genomic profiling of the sample.

### CROSS-REFERENCE TO RELATED APPLICATIONS

This application claims the priority benefit of U.S. Provisional Patent Application Ser. No. 63/292,395, filed Dec. 21, 2021, the contents of which are incorporated herein by reference in their entirety.

### FIELD OF THE INVENTION

The present disclosure relates generally to genomic profiling methods and systems, and more specifically to methods and systems for predicting the success of performing genomic profiling for individual samples based on pre-analytic variables for the individual sample.

### BACKGROUND

Genomic profiling (GP) is a next-generation sequencing (NGS)-based method that allows the analysis of a panel of genes (e.g., tens to hundreds of genes) in a single assay for the purpose of detecting genomic alterations (e.g., variant nucleic acid sequences) that may be diagnostic and/or prognostic for a disease such as cancer. For example, GP can be used to detect the four main classes of genomic alterations known to drive cancer growth: base substitutions, insertions and deletions, copy number alterations (CNAs), and rearrangements or fusions.

Despite the potential advantages and demonstrated success of using GP to identify disease-related genomic alterations, a variety of challenges to expanded implementation of GP testing remain. Existing guidelines for sample submission by physicians don't account for pre-analytical variables that may impact the success of performing GP. Thus, a method for more accurately predicting the likelihood of successfully performing GP on a specific sample would be advantageous in terms of both reducing the time and cost of performing GP, and for improving the impact of GP test results on healthcare decision making and healthcare outcomes.

### BRIEF SUMMARY OF THE INVENTION

Disclosed herein are methods and systems for more accurately predicting the likelihood of successfully performing genomic profiling (GP) on a specific sample based on a set of pre-analytic variables associated with the sample. In some instances, the disclosed methods and systems are especially useful for predicting the likelihood of successfully performing comprehensive genomic profiling (CGP) on a specific sample based on a set of pre-analytic variables associated with the sample. The methods comprise the use of a trained model to process pre-analytical data for a sample input by a physician or other healthcare provider and output an evidence-based prediction of the likelihood that a GP assay using the sample will yield reliable results. In some instances, e.g., if the predicted likelihood of performing a successful GP analysis is low, a recommendation may be made that a new sample be procured prior to submission for GP testing. The disclosed methods and systems are advantageous in terms of both reducing the time and cost of performing GP and improving the impact of GP test results on healthcare decision making and healthcare outcomes.

Disclosed herein are methods for predicting a likelihood of success for performing genomic profiling of a sample derived from a subject, comprising: receiving data for a plurality of pre-analytical variables associated with the sample; applying the received data to a multivariable model trained to predict outcomes for genomic profiling assays; generating a prediction of the likelihood of success for performing genomic profiling of the sample based on the applied multivariable model; and reporting, using the one or more processors, the prediction of the likelihood of success for performing genomic profiling of the sample.

In some embodiments, the method further comprises: based on the prediction of the likelihood of success being equal to or greater than a predefined threshold, providing a plurality of nucleic acid molecules obtained from the sample from the subject; ligating one or more adapters onto one or more nucleic acid molecules from the plurality of nucleic acid molecules; amplifying the one or more ligated nucleic acid molecules from the plurality of nucleic acid molecules; capturing amplified nucleic acid molecules from the amplified nucleic acid molecules; sequencing, by a sequencer, the captured nucleic acid molecules to obtain a plurality of sequence reads that represent the captured nucleic acid molecules and that overlap one or more gene loci within one or more subgenomic intervals in the sample; and generating, by one or more processors, a genomic profile including sequence read analysis data based on the sequence reads for the sample.

In some embodiments, the method further comprises: training the multivariable model using training data. In some embodiments, the training data comprises data derived from univariate analyses of clinical study data for samples collected from subjects representing a range of subject age, subject sex, diagnosis, stage of disease, sample type, sample collection site, sample collection method, sample preparation method, sample preservation method, sample age, sample transportation method, or any combination thereof. In some embodiments, the training data further comprises data for ECOG status, subject treatment status, tumor cellularity in the sample, tumor nuclei content in the sample, tissue surface area of the sample, tissue matrix in the sample, prior genomic profiling assay results, or any combination thereof.

In some embodiments, the plurality of pre-analytical variables comprises subject age, subject sex, diagnosis, stage of disease, sample type, sample collection site, sample collection method, sample preparation method, sample preservation method, sample age, sample transportation method, or any combination thereof. In some embodiments, the data for the plurality of pre-analytical variables is supplemented with data for tumor cellularity in the sample, tumor nuclei content in the sample, tissue surface area of the sample, tissue matrix in the sample, or any combination thereof.

In some embodiments, the multivariable model comprises a machine learning model. In some embodiments, the machine learning model comprises a supervised learning model. In some embodiments, the machine learning model comprises an unsupervised learning model. In some embodiments, the multivariable model comprises a logistic regression model, a multiple linear regression model, a random forest model, a neural network model, or a deep learning model.

In some embodiments, the prediction of the likelihood of success comprises a binary value, a percentage, or a score.

In some embodiments, the method further comprises: comparing the predicted likelihood of success to a predefined threshold; and based on a determination that the predicted likelihood of success is greater than or equal to a predefined threshold, outputting an indication that the sample from the subject is suitable for providing a genomic profile of the subject.

In some embodiments, the method further comprises: comparing the predicted likelihood of success to a predefined threshold; and based on a determination that the predicted likelihood of success is less than the predefined threshold, outputting a recommendation for collecting a new sample instead of submitting the sample for genomic profiling. In some embodiments, the method further comprises outputting a recommendation for sample type or sample collection site for the new sample. In some embodiments, the method further comprises outputting a recommendation for an alternative nucleic acid sequencing-based test method to perform. In some embodiments, the predefined threshold varies depending on sample type.

In some embodiments, the data for the plurality of pre-analytical variables is input by a user via a graphical user interface (GUI) on a display device. In some embodiments, the prediction of the likelihood of success for performing genomic profiling of the sample is reported via a graphical user interface (GUI) on a display device. In some embodiments, the graphical user interface (GUI) is displayed in a web browser.

In some embodiments, the data received for the plurality of pre-analytical variables includes data for sample type. In some embodiments, the remaining pre-analytical variables of the plurality of pre-analytical variables are selected based on the sample type. In some embodiments, the multivariable model is selected based on the sample type.

In some embodiments, the sample comprises a tissue biopsy sample, a liquid biopsy sample, or a normal control. In some embodiments, the sample is a tissue biopsy sample and comprises bone marrow. In some embodiments, the sample is a liquid biopsy sample and comprises blood, plasma, cerebrospinal fluid, sputum, stool, urine, or saliva. In some embodiments, the sample is a liquid biopsy sample and comprises circulating tumor cells (CTCs). In some embodiments, the sample is a liquid biopsy sample and comprises cell-free DNA (cfDNA), circulating tumor DNA (ctDNA), or any combination thereof.

In some embodiments, if the predicted likelihood of success is greater than or equal to the predefined threshold, the genomic profiling is performed and used to diagnose or confirm a diagnosis of disease in the subject. In some embodiments, the genomic profiling is also used to determine eligibility for therapy based on a biomarker status. In some embodiments, the disease is cancer. In some embodiments, the method further comprises selecting an anti-cancer therapy to administer to the subject based on the results of the genomic profiling. In some embodiments, the method further comprises determining an effective amount of an anti-cancer therapy to administer to the subject based on the results of the genomic profiling. In some embodiments, the method further comprises administering the anti-cancer therapy to the subject based on the results of the genomic profiling. In some embodiments, the anti-cancer therapy comprises chemotherapy, radiation therapy, immunotherapy, a targeted therapy, or surgery. In some embodiments, the cancer is a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

In some embodiments, the genomic profiling for the subject comprises obtaining results from a comprehensive genomic profiling (CGP) test, a gene expression profiling test, a cancer hotspot panel test, a DNA methylation test, a DNA fragmentation test, an RNA fragmentation test, or any combination thereof. In some embodiments, the genomic profiling for the subject further comprises obtaining results from a nucleic acid sequencing-based test. In some embodiments, the method further comprises selecting an anti-cancer agent, administering an anti-cancer agent, or applying an anti-cancer treatment to the subject based on the genomic profiling results.

Also disclosed herein are systems comprising: one or more processors; and a memory communicatively coupled to the one or more processors and configured to store instructions that, when executed by the one or more processors, cause the system to: receive the data for a plurality of pre-analytical variables associated with a sample derived from a subject; apply the received data to a multivariable model trained to predict outcomes for genomic profiling assays; generate a prediction of the likelihood of success for performing genomic profiling of the sample based on the applied multivariable model; and report the prediction of the likelihood of success for performing genomic profiling of the sample.

In some embodiments, the instructions further cause the system to compare the predicted likelihood of success to a predefined threshold; and based on a determination that the predicted likelihood of success is less than the predefined threshold, output a recommendation for collecting a new sample instead of submitting the sample for genomic profiling. In some embodiments, the data for the plurality of pre-analytical variables is input by a user via a graphical user interface (GUI) on a display device. In some embodiments, the prediction of the likelihood of success for performing genomic profiling of the sample is reported via a graphical user interface (GUI) on a display device. In some embodiments, the graphical user interface (GUI) is displayed in a web browser.

Disclosed herein are non-transitory computer-readable storage media storing one or more programs, the one or more programs comprising instructions, which when executed by one or more processors of a system, cause the system to: receive the data for a plurality of pre-analytical variables associated with a sample derived from a subject; apply the received data to a multivariable model trained to predict outcomes for genomic profiling assays; generate a prediction of the likelihood of success for performing genomic profiling of the sample based on the applied multivariable model; and report the prediction of the likelihood of success for performing genomic profiling of the sample. In some embodiments, the instructions further cause the system to compare the predicted likelihood of success to a predefined threshold; and based on a determination that the predicted likelihood of success is less than the predefined threshold, output a recommendation for collecting a new sample instead of submitting the sample for genomic profiling.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### BRIEF DESCRIPTION OF THE DRAWINGS

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawings, of which:

FIG. 1 provides a non-limiting example of a process flowchart for receiving and processing pre-analytical variable data using a multivariable model to predict a likelihood of success for performing GP on a specific sample.

FIG. 2 provides a non-limiting example of a process flowchart for displaying a request for and inputting the pre-analytical variable data for a specific sample via a graphical user interface (GUI), processing the pre-analytical variable data using a multivariable model to predict a likelihood of success for performing GP on the sample, and displaying the predicted likelihood of success for performing GP on the sample in the graphical user interface.

FIG. 3 depicts an exemplary computing device, in accordance with some instances of the systems described herein.

FIG. 4 depicts an exemplary computer system or computer network, in accordance with some instances of the systems described herein.

### DETAILED DESCRIPTION

Disclosed herein are methods and systems for more accurately predicting the likelihood of successfully performing genomic profiling (GP) on a specific sample based on a set of pre-analytic variables associated with the sample. In some instances, the disclosed methods and systems are especially useful for predicting the likelihood of successfully performing comprehensive genomic profiling (CGP) on a specific sample based on a set of pre-analytic variables associated with the sample. The methods comprise the use of a trained model to process pre-analytical data for a sample (e.g., data input by a physician or other healthcare provider) and output an evidence-based prediction of the likelihood that a GP assay using the sample will yield reliable results. In some instances, e.g., if the predicted likelihood of performing a successful GP analysis is low, a recommendation may be made that a new sample be procured prior to submission for GP testing. The disclosed methods and systems are advantageous in terms of both reducing the time and cost of performing GP and improving the impact of GP test results on healthcare decision making and healthcare outcomes.

In some instances, for example, methods are described that comprise receivingdata for a plurality of pre-analytical variables associated with the sample; applying the received data to a multivariable model trained to predict outcomes for GP assays; generating a prediction of the likelihood of success for performing GP of the sample based on the applied multivariable model; and reporting the prediction of the likelihood of success for performing GP of the sample.

Examples of pre-analytical variables include, but are not limited to, subject age, subject sex, diagnosis, stage of disease, sample type, sample collection site, sample collection method, sample preparation method, sample preservation method, sample age, sample transportation method, or any combination thereof.

In some instances, the method may further comprise: comparing the predicted likelihood of success to a predefined threshold; and based on a determination that the predicted likelihood of success is less than the predefined threshold, outputting a recommendation for collecting a new sample instead of submitting the sample for GP.

In some instances, the data for the plurality of pre-analytical variables is input by a user via a graphical user interface (GUI) on a display device. In some instances, the prediction of the likelihood of success for performing GP of the sample is reported via a graphical user interface (GUI) on a display device. In some instances, the graphical user interface (GUI) is displayed in a web browser.

### Definitions

Unless otherwise defined, all of the technical terms used herein have the same meaning as commonly understood by one of ordinary skill in the art in the field to which this disclosure belongs.

As used in this specification and the appended claims, the singular forms “a”, “an”, and “the” include plural references unless the context clearly dictates otherwise. Any reference to “or” herein is intended to encompass “and/or” unless otherwise stated.

As used herein, the terms “comprising” (and any form or variant of comprising, such as “comprise” and “comprises”), “having” (and any form or variant of having, such as “have” and “has”), “including” (and any form or variant of including, such as “includes” and “include”), or “containing” (and any form or variant of containing, such as “contains” and “contain”), are inclusive or open-ended and do not exclude additional, un-recited additives, components, integers, elements, or method steps.

As used herein, the term “subgenomic interval” (or “subgenomic sequence interval”) refers to a portion of a genomic sequence.

As used herein, the term “subject interval” refers to a subgenomic interval or an expressed subgenomic interval (e.g., the transcribed sequence of a subgenomic interval).

As used herein, the terms “variant sequence” or “variant” are used interchangeably and refer to a modified nucleic acid sequence relative to a corresponding “normal” or “wild-type” sequence. In some instances, a variant sequence may be a “short variant sequence” (or “short variant”), i.e., a variant sequence of less than about 50 base pairs in length.

The terms “allele frequency” and “allele fraction” are used interchangeably herein and refer to the fraction of sequence reads corresponding to a particular allele relative to the total number of sequence reads for a genomic locus.

The terms “variant allele frequency” and “variant allele fraction” are used interchangeably herein and refer to the fraction of sequence reads corresponding to a particular variant allele relative to the total number of sequence reads for a genomic locus.

As used herein, the term “pre-analytical variable” refers to a sample-specific characteristic or parameter related to patient history and/or sample history. Examples of pre-analytical variables include, but are not limited to, patient sex, patient age, patient diagnosis, stage of disease, sample type, sample collection site, sample preparation method, sample age, sample preservation method, sample transportation method, or any combination thereof. In some instances, pre-analytical variable data may be supplemented with additional data, e.g., Eastern Cooperative Oncology Group (ECOG) status, patient treatment status, sample imaging-based characteristics such as tumor cellularity in the sample, tumor nuclei content in the sample, tissue surface area of the sample, tissue matrix in the sample, prior GP assay results, or any combination thereof.

As used herein, the phrase “likelihood of success for performing genomic profiling” refers to a prediction of whether a nucleic-acid based analysis, including but not limited to a GP assay, personalized GP assay (e.g., a hotspot panel), or other molecular-type assay, will yield reliable results for a specific, individual sample.

The section headings used herein are for organizational purposes only and are not to be construed as limiting the subject matter described.

### Methods for Predicting Genomic Profiling Success:

The disclosed methods for predicting the likelihood of successfully performing GP on a specific sample based on a set of pre-analytic variables associated with the sample allow physicians/systems to access a prediction of the anticipated sequencing success for the specimens they have available for an individual patient, thus enabling them to decide which specimen to send in for GP testing or, alternatively, to decide if another specimen should be procured if the currently available specimen(s) are not likely to result in a successful GP testing result.

There are existing guidelines to follow when submitting samples, but these guidelines don't account for pre-analytical variables that may impact the success of performing a GP analysis, and improved methods are required. The methods described herein make use of a trained model (e.g., a multivariable regression model) that takes into account multiple pre-analytical variables and uses real-world data to produce an evidence-based success score (e.g., a prediction of the probability or likelihood of performing GP on the sample and obtaining reliable results). For example, a score equal to or greater than a threshold may indicate that the sample is suitable for performing GP (e.g., that a GP analysis of the sample would be successful).

As noted above, examples of pre-analytical variables that may impact the likelihood of success for performing GP include, but are not limited to, patient sex, patient age, patient diagnosis, sample type, sample collection site, sample preparation method, sample age, sample preservation method, sample transportation method, etc. In some instances, pre-analytical variable data may be supplemented with additional data, e.g., sample imaging-based characteristics such as tumor cellularity in the sample, tumor nuclei content in the sample, tissue surface area of the sample, tissue matrix in the sample, or any combination thereof.

In some instances, the optimal set of pre-analytical variables to consider and/or the model used for predicting the likelihood of success for performing GP may depend on the specific sample type. For example, for some tissue specimens, the set of pre-analytical variables used may comprise specimen type, patient age, patient sex, patient diagnosis, and specimen collection site, as well as additional sample characteristics such as tumor nuclei cellularity, tissue surface area, tissue matrix, etc. Alternatively, for a liquid biopsy sample, the set of pre-analytical variables used may comprise patient age, patient sex, patient diagnosis, stage of disease, etc. In some cases, pre-analytical variables may influence the likelihood of GP success as being predictors of, e.g., the amount of DNA available in the sample, the quality of the DNA available in the sample, or other biological factors. The amounts of circulating cell-free DNA (cfDNA) and circulating tumor DNA ctDNA) present in peripheral blood liquid biopsies, for example, can vary depending on several pre-analytical variables (Huang, et al. (2021) “Circulating Cell-Free DNA Yield and Circulating-Tumor DNA Quantity from Liquid Biopsies of 12,139 Cancer Patients”, Clinical Chem. October 9: hvab176. doi: 10.1093/clinchem/hvab176. Epub ahead of print. PMID: 34626187). Non-limiting examples of strong predictors for GP success include sample type, the size of a biopsy sample, or the amount of tumor nuclei visible in the sample.

In some instances, a system configured to perform the disclosed methods may be accessed by ordering physicians through, e.g., a graphical user interface displayed on a website. The physician or other healthcare provider may enter data for as many pre-analytic variables that they have available for a given specimen into the website, and the trained model process the data to generate a probability for performing a successful GP analysis. In some instances, the data for pre-analytical variables can be automatically retrieved by the system, e.g., from one or more databases.

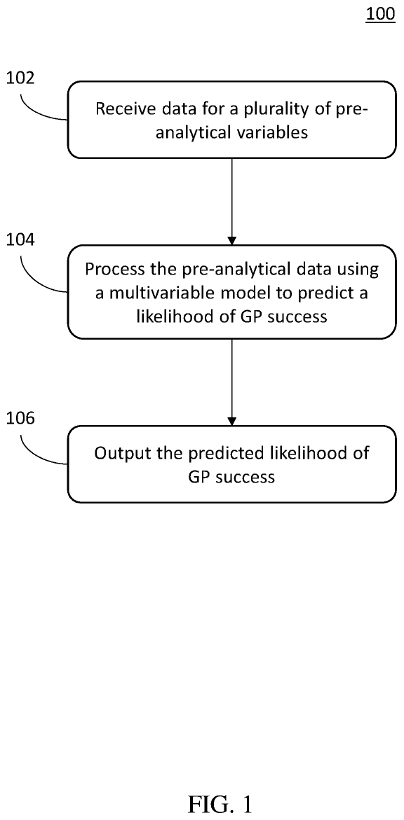

FIG. 1 provides a non-limiting example of a flowchart for a process 100 for receiving and processing pre-analytical variable data using a multivariable model to predict a likelihood of success for performing GP on a specific sample. Process 100 can be performed, for example, using one or more electronic devices (e.g., computers) implementing a software platform. In some examples, process 100 is performed using a client-server system, and the blocks of process 100 are divided up in any manner between the server and a client device. In other examples, the blocks of process 100 are divided up between the server and multiple client devices. Thus, while portions of process 100 are described herein as being performed by particular devices of a client-server system, it will be appreciated that process 100 is not so limited. In other examples, process 100 is performed using only a client device or only multiple client devices. In process 100, some blocks are, optionally, combined, the order of some blocks is, optionally, changed, and some blocks are, optionally, omitted. In some examples, additional steps may be performed in combination with the process 100. Accordingly, the operations as illustrated (and described in greater detail below) are exemplary by nature and, as such, should not be viewed as limiting.

At step 102 in FIG. 1, data for a plurality of pre-analytical variables associated with a sample are received (e.g., input by a physician or other healthcare provider or retrieved from one or more databases).

In some instances, the plurality of pre-analytical variables comprises subject age, subject sex, diagnosis, stage of disease, sample type, sample collection site, sample collection method, sample preparation method, sample preservation method, sample age, sample transportation method, or any combination thereof.

In some instances, the data for the plurality of pre-analytical variables is supplemented with data for tumor cellularity in the sample, tumor nuclei content in the sample, tissue surface area of the sample, tissue matrix in the sample, or any combination thereof.

In some instances, the number of pre-analytical variables and/or supplementary characteristics for which data is received may be 1, 2, 3, 4, 5, 6, 7, 8, 9, 10, 11, 12, 13, 14, 15, 16, 17, 18, 19, 20, or more than 20.

The sample may comprise any of a variety of sample types. For example, the sample may comprise a tissue biopsy sample, a liquid biopsy sample, and/or a normal control. In some instances, the sample is a tissue biopsy sample and comprises bone marrow. In some instances, the sample is a liquid biopsy sample and comprises blood, plasma, cerebrospinal fluid, sputum, stool, urine, or saliva. In some instances, the sample is a liquid biopsy sample and comprises circulating tumor cells (CTCs). In some instances, the sample is a liquid biopsy sample and comprises cell-free DNA (cfDNA), circulating tumor DNA (ctDNA), or any combination thereof.

At step 104 in FIG. 1, the data received for the plurality of pre-analytical variables and supplemental data is processed using a multivariable model (e.g., a multivariable regression model) to generate a prediction of the likelihood of performing a successful GP analysis on the sample (e.g., a probability or a success score). In some instances, different multivariable models (e.g., different multivariable regression models) may be used to predict the likelihood of performing a successful GP analysis for different sample types. In some instances, the prediction of the likelihood of success may comprise a binary value, a percentage, or a score.

Any of a variety of statistical analysis and/or machine learning techniques may be used to implement the multivariable model. In some instances, for example, the multivariable model may comprise a multiple linear regression model or a logistic regression analysis. In some instances, the multivariable model may comprise a machine learning model, e.g., a supervised or unsupervised learning model. In some instances, the machine learning model may comprise a random forest model, a neural network model, or a deep learning model.

In some instances, the multivariable model may be trained on training data derived from, for example, univariate analyses of clinical study data for samples collected from subjects representing a range of subject age, subject sex, diagnosis, stage of disease, sample type, sample collection site, sample collection method, sample preparation method, sample preservation method, sample age, sample transportation method, or any combination thereof.

In some instances, the training data used to train the multivariable model may further comprise data for ECOG status, treatment status, tumor cellularity in the sample, tumor nuclei content in the sample, tissue surface area of the sample, tissue matrix in the sample, prior GP assay results, or any combination thereof.

At step 106 in FIG. 1, the predicted likelihood of success for performing GP on the sample is output (e.g., reported to the ordering physician or other healthcare provider). In some instances, the process may further comprise comparing the predicted likelihood of success to a predefined threshold; and based on a determination that the predicted likelihood of success is less than the predefined threshold, outputting a recommendation for collecting a new sample instead of submitting the sample for GP. In some instances, the process may further comprise outputting a recommendation for sample type or sample collection site for the new sample. In some instances, the process may further comprise outputting a recommendation for an alternative nucleic acid sequencing-based test method to perform.

In some instances, the predefined threshold may vary depending on sample type. For example, the predefined threshold may be different for tissue samples and liquid biopsy samples. In some instances, the threshold may be determined empirically by evaluating data for GP result-based healthcare decisions as a function of candidate thresholds for multiple samples. In some instances, the value of the predefined threshold (i.e., a “probability of success” threshold) for a given sample type may be 40%, 45%, 50%, 55%, 60%, 65%, 70%, 75%, 80%, 85%, 90%, or 95%, where if the predicted likelihood of success for a give sample is less than the predefined threshold, a recommendation is made to collect a new sample. Alternatively, or additionally, the predefined threshold may be determined and/or modified based on a success rate of one or more prior processes in which a new sample was obtained.

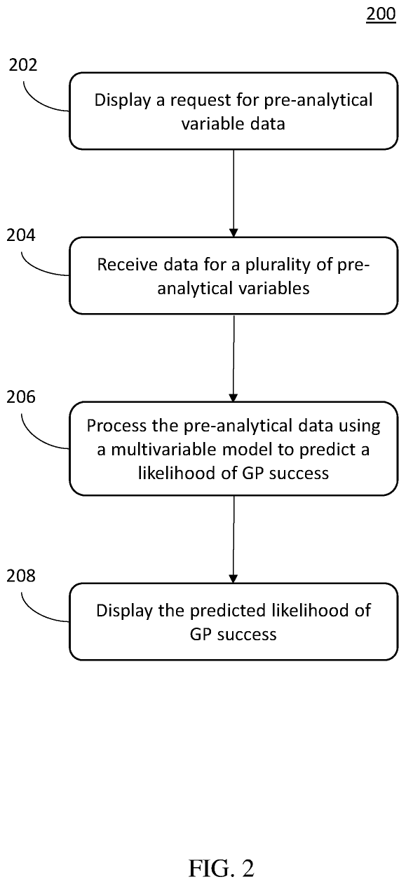

FIG. 2 provides a non-limiting example of a flowchart for a process 200 for displaying a request for and inputting the pre-analytical variable data for a specific sample via a graphical user interface (GUI), processing the pre-analytical variable data using a multivariable model to predict a likelihood of success for performing GP on the sample, and displaying the predicted likelihood of success for performing GP on the sample in the graphical user interface.

At step 202 in FIG. 2, one or more fields in a GUI are displayed to request input of data for a plurality of pre-analytical variables and/or supplementary data associated with an individual sample. In some instances, the GUI may comprise additional fields for entry of, e.g., the ordering physician's name, the ordering physician's affiliation and/or address, patient name, patient billing address, patient insurance information, etc. In some instances, the list of requested pre-analytical variable data is updated once an entry for sample type has been made by the user (e.g., a physician or other healthcare provider).

At step 204 in FIG. 2, the requested information (e.g., data for a plurality of pre-analytical variables and/or supplementary data associated with the sample) is input by a physician or other healthcare provider. In some instances, data for two or more of the plurality of pre-analytical variables (and/or supplementary data) may be entered within a single GUI field. In some instances, data for each pre-analytical variable (or item of supplementary data) may be entered into a separate field. In some instances, the requested information (e.g., data for a plurality of pre-analytical variables and/or supplementary data associated with the sample) may be downloaded from a database (e.g., a cloud-based database) which contains the patient's medical records upon submission of a read request. In some instances, the downloaded data may be used to autofill the data fields in a GUI.

At step 206 in FIG. 2, the data entered for the plurality of pre-analytical variables and/or supplementary data associated with the sample is processed using a multivariable model (e.g., a multivariable regression model) to generate a prediction of the likelihood of performing a successful GP analysis of the sample. In some instances, a choice of multivariable model (i.e., selected from a plurality of trained multivariable models) may be made based on a sample type entered by the user (e.g., a physician or other healthcare provider) and/or based on the specific subset of pre-analytical variables for which data has been entered by the user.

As noted above, any of a variety of statistical analysis and/or machine learning techniques may be used to implement the multivariable model. In some instances, for example, the multivariable model may comprise a multiple linear regression model or a logistic regression analysis. In some instances, the multivariable model may comprise a machine learning model, e.g., a supervised or unsupervised learning model. In some instances, the machine learning model may comprise a random forest model, a neural network model, or a deep learning model.

In some instances, the multivariable model may be trained on training data derived from, for example, univariate analyses of clinical study data for samples collected from subjects representing a range of subject age, subject sex, diagnosis, stage of disease, sample type, sample collection site, sample collection method, sample preparation method, sample preservation method, sample age, sample transportation method, or any combination thereof.

In some instances, the training data used to train the multivariable model may further comprise data for ECOG status, treatment status, tumor cellularity in the sample, tumor nuclei content in the sample, tissue surface area of the sample, tissue matrix in the sample, prior GP assay results, or any combination thereof.

In some instances, the multivariable model may be trained using one or more sets of training data and any of a variety of machine learning training methodologies (e.g., a gradient descent method, a Newton method, a conjugate gradient method, a quasi-Newton method, or a Levenberg-Marquardt method, and the like).

At step 208 in FIG. 2, the predicted likelihood of GP success is displayed in a GUI field. As noted above, in some instances, the process may further comprise comparing the predicted likelihood of success to a predefined threshold; and based on a determination that the predicted likelihood of success is less than the predefined threshold, displaying a recommendation for collecting a new sample instead of submitting the sample for GP. In some instances, the process may further comprise displaying a recommendation for sample type or sample collection site for the new sample. In some instances, the process may further comprise displaying a recommendation for an alternative nucleic acid sequencing-based test method to perform. In some instances, the process may comprise displaying multiple alternative recommendations.

### Methods of Use

In some instances, the disclosed methods may further comprise one or more of the steps of: (i) obtaining the sample from the subject (e.g., a subject suspected of having or determined to have cancer), (ii) extracting nucleic acid molecules (e.g., a mixture of tumor nucleic acid molecules and non-tumor nucleic acid molecules) from the sample, (iii) ligating one or more adapters to the nucleic acid molecules extracted from the sample (e.g., one or more amplification primers, flow cell adaptor sequences, substrate adapter sequences, or sample index sequences), (iv) amplifying the nucleic acid molecules (e.g., using a polymerase chain reaction (PCR) amplification technique, a non-PCR amplification technique, or an isothermal amplification technique), (v) capturing nucleic acid molecules from the amplified nucleic acid molecules (e.g., by hybridization to one or more bait molecules, where the bait molecules each comprise one or more nucleic acid molecules that each comprising a region that is complementary to a region of a captured nucleic acid molecule), (vi) sequencing the nucleic acid molecules extracted from the sample (or library proxies derived therefrom) using, e.g., a next-generation (massively parallel) sequencing technique, a whole genome sequencing (WGS) technique, a whole exome sequencing technique, a targeted sequencing technique, a direct sequencing technique, or a Sanger sequencing technique) using, e.g., a next-generation (massively parallel) sequencer, and (vii) generating, displaying, transmitting, and/or delivering a report (e.g., an electronic, web-based, or paper report) to the subject (or patient), a caregiver, a healthcare provider, a physician, an oncologist, an electronic medical record system, a hospital, a clinic, a third-party payer, an insurance company, or a government office. In some instances, the report comprises output from the methods described herein. In some instances, all or a portion of the report may be displayed in the graphical user interface of an online or web-based healthcare portal. In some instances, the report is transmitted via a computer network or peer-to-peer connection.

The disclosed methods may be used with any of a variety of samples. For example, in some instances, the sample may comprise a tissue biopsy sample, a liquid biopsy sample, or a normal control. In some instances, the sample may be a liquid biopsy sample and may comprise blood, plasma, cerebrospinal fluid, sputum, stool, urine, or saliva. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the nucleic acid molecules extracted from a sample may comprise a mixture of tumor nucleic acid molecules and non-tumor nucleic acid molecules. In some instances, the tumor nucleic acid molecules may be derived from a tumor portion of a heterogeneous tissue biopsy sample, and the non-tumor nucleic acid molecules may be derived from a normal portion of the heterogeneous tissue biopsy sample. In some instances, the sample may comprise a liquid biopsy sample, and the tumor nucleic acid molecules may be derived from a circulating tumor DNA (ctDNA) fraction of the liquid biopsy sample while the non-tumor nucleic acid molecules may be derived from a non-tumor, cell-free DNA (cfDNA) fraction of the liquid biopsy sample.

In some instances, the disclosed methods for predicting GP success may ensure that reliable GP results are obtained, which may then be used to diagnose the presence of disease or other condition (e.g., cancer, genetic disorders (such as Down Syndrome and Fragile X), neurological disorders, or any other disease type where detection of variants, e.g., copy number alternations, are relevant to diagnosing, treating, or predicting said disease) in a subject (e.g., a patient). In some instances, the disclosed methods may be applicable to diagnosis of any of a variety of cancers as described elsewhere herein.

In some instances, the disclosed methods for predicting GP success may ensure that reliable GP results are obtained, which may then be used to predict genetic disorders in fetal DNA. (e.g., for invasive or non-invasive prenatal testing). For example, sequence read data obtained sequencing fetal DNA extracted from samples obtained using invasive amniocentesis, chorionic villus sampling (cVS), or fetal umbilical cord sampling techniques, or obtained using non-invasive sampling of cell-free DNA (cfDNA) samples (which comprises a mix of maternal cfDNA and fetal cfDNA), may be processed according to the disclosed methods to identify variants, e.g., copy number alterations, associated with, e.g., Down Syndrome (trisomy 21), trisomy 18, trisomy 13, and extra or missing copies of the X and Y chromosomes.

In some instances, the disclosed methods for predicting GP success may ensure that reliable GP results are obtained, which may then be used to select a subject (e.g., a patient) for a clinical trial. In some instances, patient selection for clinical trials based on, e.g., identification of one or more variants at one or more gene loci, may accelerate the development of targeted therapies and improve the healthcare outcomes for treatment decisions.

In some instances, the disclosed methods for predicting GP success may ensure that reliable GP results are obtained, which may then be used to select an appropriate therapy or treatment (e.g., an anti-cancer therapy or anti-cancer treatment) for a subject. In some instances, for example, the anti-cancer therapy or treatment may comprise use of a poly (ADP-ribose) polymerase inhibitor (PARPi), a platinum compound, chemotherapy, radiation therapy, a targeted therapy (e.g., immunotherapy), surgery, or any combination thereof.

In some instances, the disclosed methods for predicting GP success may ensure that reliable GP results are obtained, which may then be used in treating a disease (e.g., a cancer) in a subject. For example, in response to identifying the presence of a variant sequence, using a GP assay, an effective amount of an anti-cancer therapy or anti-cancer treatment may be administered to the subject.

In some instances, the disclosed methods for predicting GP success may ensure that reliable GP results are obtained, which may then be used for monitoring disease progression or recurrence (e.g., cancer or tumor progression or recurrence) in a subject. For example, in some instances, the GP assay may be used to detect the presence of one or more variant sequences in a first sample obtained from the subject at a first time point, and used to detect the presence of one or more variant sequences in a second sample obtained from the subject at a second time point, where comparison of the first determination and the second determination allows one to monitor disease progression or recurrence. In some instances, the first time point is chosen before the subject has been administered a therapy or treatment, and the second time point is chosen after the subject has been administered the therapy or treatment.

In some instances, the GP analysis may be used for adjusting a therapy or treatment (e.g., an anti-cancer treatment or anti-cancer therapy) for a subject, e.g., by adjusting a treatment dose and/or selecting a different treatment in response to a change in a variant allele frequency detected in a sample derived from the subject.

In some instances, the value of predicting GP success is to ensure that reliable GP results are obtained, which may then be used as a prognostic or diagnostic indicator associated with the sample. For example, in some instances, the prognostic or diagnostic indicator may comprise an indicator of the presence of a disease (e.g., cancer) in the sample, an indicator of the probability that a disease (e.g., cancer) is present in the sample, an indicator of the probability that the subject from which the sample was derived will develop a disease (e.g., cancer) (i.e., a risk factor), or an indicator of the likelihood that the subject from which the sample was derived will respond to a particular therapy or treatment.

In some instances, the disclosed methods for predicting GP success may ensure that reliable GP results are obtained. A GP process may comprise identification of the presence of variant sequences at one or more gene loci in a sample derived from a subject as part of detecting, monitoring, predicting a risk factor, or selecting a treatment for a particular disease, e.g., cancer. In some instances, the variant panel selected for GP may comprise the detection of variant sequences at a selected set of gene loci. In some instances, the variant panel selected for GP may comprise detection of variant sequences at a number of gene loci through GP, a next-generation sequencing (NGS) approach used to assess hundreds of genes (including relevant cancer biomarkers) in a single assay. Improved reliability of GP results, as facilitated using the disclosed methods for predicting GP success, can improve the validity of, e.g., disease detection calls and treatment decisions, made on the basis of the genomic profile by, for example, independently confirming the presence of a variant sequence in a given patient sample.

In some instances, a genomic profile may comprise information on the presence of genes (or variant sequences thereof), copy number variations, epigenetic traits, proteins (or modifications thereof), and/or other biomarkers in an individual's genome and/or proteome, as well as information on the individual's corresponding phenotypic traits and the interaction between genetic or genomic traits, phenotypic traits, and environmental factors.

In some instances, a genomic profile for the subject may comprise results from a GP test, a nucleic acid sequencing-based test, a gene expression profiling test, a cancer hotspot panel test, a DNA methylation test, a DNA fragmentation test, an RNA fragmentation test, or any combination thereof.

In some instances, the method can further include administering or applying a treatment or therapy (e.g., an anti-cancer agent, anti-cancer treatment, or anti-cancer therapy) to the subject based on the generated genomic profile. An anti-cancer agent or anti-cancer treatment may refer to a compound that is effective in the treatment of cancer cells. Examples of anti-cancer agents or anti-cancer therapies include, but not limited to, alkylating agents, antimetabolites, natural products, hormones, chemotherapy, radiation therapy, immunotherapy, surgery, or a therapy configured to target a defect in a specific cell signaling pathway, e.g., a defect in a DNA mismatch repair (MMR) pathway.

### Samples

The disclosed methods and systems may be used with any of a variety of samples (also referred to herein as specimens) comprising nucleic acids (e.g., DNA or RNA) that are collected from a subject (e.g., a patient). Examples include, but are not limited to, a tumor sample, a tissue sample, a biopsy sample, a blood sample (e.g., a peripheral whole blood sample), a blood plasma sample, a blood serum sample, a lymph sample, a saliva sample, a sputum sample, a urine sample, a gynecological fluid sample, a circulating tumor cell (CTC) sample, a cerebral spinal fluid (CSF) sample, a pericardial fluid sample, a pleural fluid sample, an ascites (peritoneal fluid) sample, a feces (or stool) sample, or other body fluid, secretion, and/or excretion sample (or cell sample derived therefrom). In certain instances, the sample may be frozen sample or a formalin-fixed paraffin-embedded (FFPE) sample.

In some instances, the sample may be collected by tissue resection (e.g., surgical resection), needle biopsy, bone marrow biopsy, bone marrow aspiration, skin biopsy, endoscopic biopsy, fine needle aspiration, oral swab, nasal swab, vaginal swab or a cytology smear, scrapings, washings or lavages (such as a ductal lavages or bronchoalveolar lavages), etc.

In some instances, the sample is a liquid biopsy sample, and may comprise, e.g., whole blood, blood plasma, blood serum, urine, stool, sputum, saliva, or cerebrospinal fluid. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the sample may comprise one or more premalignant or malignant cells. Premalignant, as used herein, refers to a cell or tissue that is not yet malignant but is poised to become malignant. In certain instances, the sample may be acquired from a solid tumor, a soft tissue tumor, or a metastatic lesion. In certain instances, the sample may be acquired from a hematologic malignancy or pre-malignancy. In other instances, the sample may comprise a tissue or cells from a surgical margin. In certain instances, the sample may comprise tumor-infiltrating lymphocytes. In some instances, the sample may comprise one or more non-malignant cells. In some instances, the sample may be, or is part of, a primary tumor or a metastasis (e.g., a metastasis biopsy sample). In some instances, the sample may be obtained from a site (e.g., a tumor site) with the highest percentage of tumor (e.g., tumor cells) as compared to adjacent sites (e.g., sites adjacent to the tumor). In some instances, the sample may be obtained from a site (e.g., a tumor site) with the largest tumor focus (e.g., the largest number of tumor cells as visualized under a microscope) as compared to adjacent sites (e.g., sites adjacent to the tumor).

In some instances, the disclosed methods may further comprise analyzing a primary control (e.g., a normal tissue sample). In some instances, the disclosed methods may further comprise determining if a primary control is available and, if so, isolating a control nucleic acid (e.g., DNA) from said primary control. In some instances, the sample may comprise any normal control (e.g., a normal adjacent tissue (NAT)) if no primary control is available. In some instances, the sample may be or may comprise histologically normal tissue. In some instances, the method includes evaluating a sample, e.g., a histologically normal sample (e.g., from a surgical tissue margin) using the methods described herein. In some instances, the disclosed methods may further comprise acquiring a sub-sample enriched for non-tumor cells, e.g., by macro-dissecting non-tumor tissue from said NAT in a sample not accompanied by a primary control. In some instances, the disclosed methods may further comprise determining that no primary control and no NAT is available, and marking said sample for analysis without a matched control.

In some instances, samples obtained from histologically normal tissues (e.g., otherwise histologically normal surgical tissue margins) may still comprise a genetic alteration such as a variant sequence as described herein. The methods may thus further comprise re-classifying a sample based on the presence of the detected genetic alteration. In some instances, multiple samples (e.g., from different subjects) are processed simultaneously.

The disclosed methods and systems may be applied to the analysis of nucleic acids extracted from any of variety of tissue samples (or disease states thereof), e.g., solid tissue samples, soft tissue samples, metastatic lesions, or liquid biopsy samples. Examples of tissues include, but are not limited to, connective tissue, muscle tissue, nervous tissue, epithelial tissue, and blood. Tissue samples may be collected from any of the organs within an animal or human body. Examples of human organs include, but are not limited to, the brain, heart, lungs, liver, kidneys, pancreas, spleen, thyroid, mammary glands, uterus, prostate, large intestine, small intestine, bladder, bone, skin, etc.

In some instances, the nucleic acids extracted from the sample may comprise deoxyribonucleic acid (DNA) molecules. Examples of DNA that may be suitable for analysis by the disclosed methods include, but are not limited to, genomic DNA or fragments thereof, mitochondrial DNA or fragments thereof, cell-free DNA (cfDNA), and circulating tumor DNA (ctDNA). Cell-free DNA (cfDNA) is comprised of fragments of DNA that are released from normal and/or cancerous cells during apoptosis and necrosis, and circulate in the blood stream and/or accumulate in other bodily fluids. Circulating tumor DNA (ctDNA) is comprised of fragments of DNA that are released from cancerous cells and tumors that circulate in the blood stream and/or accumulate in other bodily fluids.

In some instances, DNA is extracted from nucleated cells from the sample. In some instances, a sample may have a low nucleated cellularity, e.g., when the sample is comprised mainly of erythrocytes, lesional cells that contain excessive cytoplasm, or tissue with fibrosis. In some instances, a sample with low nucleated cellularity may require more, e.g., greater, tissue volume for DNA extraction.

In some instances, the nucleic acids extracted from the sample may comprise ribonucleic acid (RNA) molecules. Examples of RNA that may be suitable for analysis by the disclosed methods include, but are not limited to, total cellular RNA, total cellular RNA after depletion of certain abundant RNA sequences (e.g., ribosomal RNAs), cell-free RNA (cfRNA), messenger RNA (mRNA) or fragments thereof, the poly(A)-tailed mRNA fraction of the total RNA, ribosomal RNA (rRNA) or fragments thereof, transfer RNA (tRNA) or fragments thereof, and mitochondrial RNA or fragments thereof. In some instances, RNA may be extracted from the sample and converted to complementary DNA (cDNA) using, e.g., a reverse transcription reaction. In some instances, the cDNA is produced by random-primed cDNA synthesis methods. In other instances, the cDNA synthesis is initiated at the poly(A) tail of mature mRNAs by priming with oligo (dT)-containing oligonucleotides. Methods for depletion, poly(A) enrichment, and cDNA synthesis are well known to those of skill in the art.

In some instances, the sample may comprise a tumor content, e.g., comprising tumor cells or tumor cell nuclei. In some instances, the sample may comprise a tumor content of at least 5-50%, 10-40%, 15-25%, or 20-30% tumor cell nuclei. In some instances, the sample may comprise a tumor content of at least 5%, at least 10%, at least 20%, at least 30%, at least 40%, or at least 50% tumor cell nuclei. In some instances, the percent tumor cell nuclei is determined (e.g., calculated) by dividing the number of tumor cells in the sample by the total number of all cells within the sample that have nuclei. In some instances, for example when the sample is a liver sample comprising hepatocytes, a different tumor content calculation may be required due to the presence of hepatocytes having nuclei with twice, or more than twice, the DNA content of other, e.g., non-hepatocyte, somatic cell nuclei. In some instances, the sensitivity of detection of a genetic alteration, e.g., a variant sequence, or a determination of, e.g., microsatellite instability, may depend on the tumor content of the sample. For example, a sample having a lower tumor content can result in lower sensitivity of detection for a given size sample.

In some instances, as noted above, the sample comprises nucleic acid (e.g., DNA, RNA (or a cDNA derived from the RNA), or both), e.g., from a tumor or from normal tissue. In certain instances, the sample may further comprise a non-nucleic acid component, e.g., cells, protein, carbohydrate, or lipid, e.g., from the tumor or normal tissue.

### Subjects

In some instances, the sample is obtained (e.g., collected) from a subject (e.g., patient) with a condition or disease (e.g., a hyperproliferative disease or a non-cancer indication) or suspected of having the condition or disease. In some instances, the hyperproliferative disease is a cancer. In some instances, the cancer is a solid tumor or a metastatic form thereof. In some instances, the cancer is a hematological cancer, e.g. a leukemia or lymphoma.

In some instances, the subject has a cancer or is at risk of having a cancer. For example, in some instances, the subject has a genetic predisposition to a cancer (e.g., having a genetic mutation that increases his or her baseline risk for developing a cancer). In some instances, the subject has been exposed to an environmental perturbation (e.g., radiation or a chemical) that increases his or her risk for developing a cancer. In some instances, the subject is in need of being monitored for development of a cancer. In some instances, the subject is in need of being monitored for cancer progression or regression, e.g., after being treated with an anti-cancer therapy (or anti-cancer treatment). In some instances, the subject is in need of being monitored for relapse of cancer. In some instances, the subject is in need of being monitored for minimum residual disease (MRD). In some instances, the subject has been, or is being treated, for cancer. In some instances, the subject has not been treated with an anti-cancer therapy (or anti-cancer treatment).

In some instances, the subject (e.g., a patient) is being treated, or has been previously treated, with one or more targeted therapies. In some instances, e.g., for a patient who has been previously treated with a targeted therapy, a post-targeted therapy sample (e.g., specimen) is obtained (e.g., collected). In some instances, the post-targeted therapy sample is a sample obtained after the completion of the targeted therapy.

In some instances, the patient has not been previously treated with a targeted therapy. In some instances, e.g., for a patient who has not been previously treated with a targeted therapy, the sample comprises a resection, e.g., an original resection, or a resection following recurrence (e.g., following a disease recurrence post-therapy).

### Cancers

In some instances, the sample is acquired from a subject having a cancer. Exemplary cancers include, but are not limited to, B cell cancer (e.g., multiple myeloma), melanomas, breast cancer, lung cancer (such as non-small cell lung carcinoma or NSCLC), bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, adenocarcinomas, inflammatory myofibroblastic tumors, gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancers, essential thrombocythemia, agnogenic myeloid metaplasia, hypereosinophilic syndrome, systemic mastocytosis, familiar hypereosinophilia, chronic eosinophilic leukemia, neuroendocrine cancers, carcinoid tumors, and the like.

In some instances, the cancer is a hematologic malignancy (or premaligancy). As used herein, a hematologic malignancy refers to a tumor of the hematopoietic or lymphoid tissues, e.g., a tumor that affects blood, bone marrow, or lymph nodes. Exemplary hematologic malignancies include, but are not limited to, leukemia (e.g., acute lymphoblastic leukemia (ALL), acute myeloid leukemia (AML), chronic lymphocytic leukemia (CLL), chronic myelogenous leukemia (CML), hairy cell leukemia, acute monocytic leukemia (AMoL), chronic myelomonocytic leukemia (CMML), juvenile myelomonocytic leukemia (JMML), or large granular lymphocytic leukemia), lymphoma (e.g., AIDS-related lymphoma, cutaneous T-cell lymphoma, Hodgkin lymphoma (e.g., classical Hodgkin lymphoma or nodular lymphocyte-predominant Hodgkin lymphoma), mycosis fungoides, non-Hodgkin lymphoma (e.g., B-cell non-Hodgkin lymphoma (e.g., Burkitt lymphoma, small lymphocytic lymphoma (CLL/SLL), diffuse large B-cell lymphoma, follicular lymphoma, immunoblastic large cell lymphoma, precursor B-lymphoblastic lymphoma, or mantle cell lymphoma) or T-cell non-Hodgkin lymphoma (mycosis fungoides, anaplastic large cell lymphoma, or precursor T-lymphoblastic lymphoma)), primary central nervous system lymphoma, Sézary syndrome, Waldenström macroglobulinemia), chronic myeloproliferative neoplasm, Langerhans cell histiocytosis, multiple myeloma/plasma cell neoplasm, myelodysplastic syndrome, or myelodysplastic/myeloproliferative neoplasm.

### Nucleic Acid Extraction and Processing

DNA or RNA may be extracted from tissue samples, biopsy samples, blood samples, or other bodily fluid samples using any of a variety of techniques known to those of skill in the art (see, e.g., Example 1 of International Patent Application Publication No. WO 2012/092426; Tan, et al. (2009), “DNA, RNA, and Protein Extraction: The Past and The Present”, J. Biomed. Biotech. 2009:574398; the technical literature for the Maxwell® 16 LEV Blood DNA Kit (Promega Corporation, Madison, WI); and the Maxwell 16 Buccal Swab LEV DNA Purification Kit Technical Manual (Promega Literature #TM333, Jan. 1, 2011, Promega Corporation, Madison, WI)). Protocols for RNA isolation are disclosed in, e.g., the Maxwell® 16 Total RNA Purification Kit Technical Bulletin (Promega Literature #TB351, August 2009, Promega Corporation, Madison, WI).

A typical DNA extraction procedure, for example, comprises (i) collection of the fluid sample, cell sample, or tissue sample from which DNA is to be extracted, (ii) disruption of cell membranes (i.e., cell lysis), if necessary, to release DNA and other cytoplasmic components, (iii) treatment of the fluid sample or lysed sample with a concentrated salt solution to precipitate proteins, lipids, and RNA, followed by centrifugation to separate out the precipitated proteins, lipids, and RNA, and (iv) purification of DNA from the supernatant to remove detergents, proteins, salts, or other reagents used during the cell membrane lysis step.

Disruption of cell membranes may be performed using a variety of mechanical shear (e.g., by passing through a French press or fine needle) or ultrasonic disruption techniques. The cell lysis step often comprises the use of detergents and surfactants to solubilize lipids the cellular and nuclear membranes. In some instances, the lysis step may further comprise use of proteases to break down protein, and/or the use of an RNase for digestion of RNA in the sample.

Examples of suitable techniques for DNA purification include, but are not limited to, (i) precipitation in ice-cold ethanol or isopropanol, followed by centrifugation (precipitation of DNA may be enhanced by increasing ionic strength, e.g., by addition of sodium acetate), (ii) phenol-chloroform extraction, followed by centrifugation to separate the aqueous phase containing the nucleic acid from the organic phase containing denatured protein, and (iii) solid phase chromatography where the nucleic acids adsorb to the solid phase (e.g., silica or other) depending on the pH and salt concentration of the buffer.

In some instances, cellular and histone proteins bound to the DNA may be removed either by adding a protease or by having precipitated the proteins with sodium or ammonium acetate, or through extraction with a phenol-chloroform mixture prior to a DNA precipitation step.

In some instances, DNA may be extracted using any of a variety of suitable commercial DNA extraction and purification kits. Examples include, but are not limited to, the QIAamp (for isolation of genomic DNA from human samples) and DNAeasy (for isolation of genomic DNA from animal or plant samples) kits from Qiagen (Germantown, MD) or the Maxwell® and ReliaPrep™ series of kits from Promega (Madison, WI).

As noted above, in some instances the sample may comprise a formalin-fixed (also known as formaldehyde-fixed, or paraformaldehyde-fixed), paraffin-embedded (FFPE) tissue preparation. For example, the FFPE sample may be a tissue sample embedded in a matrix, e.g., an FFPE block. Methods to isolate nucleic acids (e.g., DNA) from formaldehyde- or paraformaldehyde-fixed, paraffin-embedded (FFPE) tissues are disclosed in, e.g., Cronin, et al., (2004) Am J Pathol. 164 (1): 35-42; Masuda, et al., (1999) Nucleic Acids Res. 27 (22): 4436-4443; Specht, et al., (2001) Am J Pathol. 158 (2): 419-429; the Ambion RecoverAll™ Total Nucleic Acid Isolation Protocol (Ambion, Cat. No. AM1975, September 2008); the Maxwell® 16 FFPE Plus LEV DNA Purification Kit Technical Manual (Promega Literature #TM349, February 2011); the E.Z.N.A.® FFPE DNA Kit Handbook (OMEGA bio-tek, Norcross, GA, product numbers D3399-00, D3399-01, and D3399-02, June 2009); and the QIAamp® DNA FFPE Tissue Handbook (Qiagen, Cat. No. 37625, October 2007). For example, the RecoverAll™ Total Nucleic Acid Isolation Kit uses xylene at elevated temperatures to solubilize paraffin-embedded samples and a glass-fiber filter to capture nucleic acids. The Maxwell® 16 FFPE Plus LEV DNA Purification Kit is used with the Maxwell® 16 Instrument for purification of genomic DNA from 1 to 10 μm sections of FFPE tissue. DNA is purified using silica-clad paramagnetic particles (PMPs), and eluted in low elution volume. The E.Z.N.A.® FFPE DNA Kit uses a spin column and buffer system for isolation of genomic DNA. QIAamp® DNA FFPE Tissue Kit uses QIAamp® DNA Micro technology for purification of genomic and mitochondrial DNA.

In some instances, the disclosed methods may further comprise determining or acquiring a yield value for the nucleic acid extracted from the sample and comparing the determined value to a reference value. For example, if the determined or acquired value is less than the reference value, the nucleic acids may be amplified prior to proceeding with library construction. In some instances, the disclosed methods may further comprise determining or acquiring a value for the size (or average size) of nucleic acid fragments in the sample, and comparing the determined or acquired value to a reference value, e.g., a size (or average size) of at least 100, 200, 300, 400, 500, 600, 700, 800, 900, or 1000 base pairs (bps). In some instances, one or more parameters described herein may be adjusted or selected in response to this determination.

After isolation, the nucleic acids are typically dissolved in a slightly alkaline buffer, e.g., Tris-EDTA (TE) buffer, or in ultra-pure water. In some instances, the isolated nucleic acids (e.g., genomic DNA) may be fragmented or sheared by using any of a variety of techniques known to those of skill in the art. For example, genomic DNA can be fragmented by physical shearing methods, enzymatic cleavage methods, chemical cleavage methods, and other methods known to those of skill in the art. Methods for DNA shearing are described in Example 4 in International Patent Application Publication No. WO 2012/092426. In some instances, alternatives to DNA shearing methods can be used to avoid a ligation step during library preparation.

### Library Preparation

In some instances, the nucleic acids isolated from the sample may be used to construct a library (e.g., a nucleic acid library as described herein). In some instances, the nucleic acids are fragmented using any of the methods described above, optionally subjected to repair of chain end damage, and optionally ligated to synthetic adapters, primers, and/or barcodes (e.g., amplification primers, sequencing adapters, flow cell adapters, substrate adapters, sample barcodes or indexes, and/or unique molecular identifier sequences), size-selected (e.g., by preparative gel electrophoresis), and/or amplified (e.g., using PCR, a non-PCR amplification technique, or an isothermal amplification technique). In some instances, the fragmented and adapter-ligated group of nucleic acids is used without explicit size selection or amplification prior to hybridization-based selection of target sequences. In some instances, the nucleic acid is amplified by any of a variety of specific or non-specific nucleic acid amplification methods known to those of skill in the art. In some instances, the nucleic acids are amplified, e.g., by a whole-genome amplification method such as random-primed strand-displacement amplification. Examples of nucleic acid library preparation techniques for next-generation sequencing are described in, e.g., van Dijk, et al. (2014), Exp. Cell Research 322:12-20, and Illumina's genomic DNA sample preparation kit.

In some instances, the resulting nucleic acid library may contain all or substantially all of the complexity of the genome. The term “substantially all” in this context refers to the possibility that there can in practice be some unwanted loss of genome complexity during the initial steps of the procedure. The methods described herein also are useful in cases where the nucleic acid library comprises a portion of the genome, e.g., where the complexity of the genome is reduced by design. In some instances, any selected portion of the genome can be used with a method described herein. For example, in certain embodiments, the entire exome or a subset thereof is isolated. In some instances, the library may include at least 95%, 90%, 80%, 70%, 60%, 50%, 40%, 30%, 20%, 10%, or 5% of the genomic DNA. In some instances, the library may consist of cDNA copies of genomic DNA that includes copies of at least 95%, 90%, 80%, 70%, 60%, 50%, 40%, 30%, 20%, 10%, or 5% of the genomic DNA. In certain instances, the amount of nucleic acid used to generate the nucleic acid library may be less than 5 micrograms, less than 1 microgram, less than 500 ng, less than 200 ng, less than 100 ng, less than 50 ng, less than 10 ng, less than 5 ng, or less than 1 ng.

In some instances, a library (e.g., a nucleic acid library) includes a collection of nucleic acid molecules. As described herein, the nucleic acid molecules of the library can include a target nucleic acid molecule (e.g., a tumor nucleic acid molecule, a reference nucleic acid molecule and/or a control nucleic acid molecule; also referred to herein as a first, second and/or third nucleic acid molecule, respectively). The nucleic acid molecules of the library can be from a single subject or individual. In some instances, a library can comprise nucleic acid molecules derived from more than one subject (e.g., 2, 3, 4, 5, 6, 7, 8, 9, 10, 20, 30 or more subjects). For example, two or more libraries from different subjects can be combined to form a library having nucleic acid molecules from more than one subject (where the nucleic acid molecules derived from each subject are optionally ligated to a unique sample barcode corresponding to a specific subject). In some instances, the subject is a human having, or at risk of having, a cancer or tumor.

In some instances, the library (or a portion thereof) may comprise one or more subgenomic intervals. In some instances, a subgenomic interval can be a single nucleotide position, e.g., a nucleotide position for which a variant at the position is associated (positively or negatively) with a tumor phenotype. In some instances, a subgenomic interval comprises more than one nucleotide position. Such instances include sequences of at least 2, 5, 10, 50, 100, 150, 250, or more than 250 nucleotide positions in length. Subgenomic intervals can comprise, e.g., one or more entire genes (or portions thereof), one or more exons or coding sequences (or portions thereof), one or more introns (or portion thereof), one or more microsatellite region (or portions thereof), or any combination thereof. A subgenomic interval can comprise all or a part of a fragment of a naturally occurring nucleic acid molecule, e.g., a genomic DNA molecule. For example, a subgenomic interval can correspond to a fragment of genomic DNA which is subjected to a sequencing reaction. In some instances, a subgenomic interval is a continuous sequence from a genomic source. In some instances, a subgenomic interval includes sequences that are not contiguous in the genome, e.g., subgenomic intervals in cDNA can include exon-exon junctions formed as a result of splicing. In some instances, the subgenomic interval comprises a tumor nucleic acid molecule. In some instances, the subgenomic interval comprises a non-tumor nucleic acid molecule.

### Targeting Gene Loci for Analysis

The methods described herein can be used in combination with, or as part of, a method for evaluating a plurality or set of subject intervals (e.g., target sequences), e.g., from a set of genomic loci (e.g., gene loci or fragments thereof), as described herein.

In some instances, the set of genomic loci evaluated by the disclosed methods comprises a plurality of, e.g., genes, which in mutant form, are associated with an effect on cell division, growth or survival, or are associated with a cancer, e.g., a cancer described herein.

In some instances, the set of gene loci evaluated by the disclosed methods comprises at least 1, at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, or more than 100 gene loci.

In some instances, the selected gene loci (also referred to herein as target gene loci or target sequences), or fragments thereof, may include subject intervals comprising non-coding sequences, coding sequences, intragenic regions, or intergenic regions of the subject genome. For example, the subject intervals can include a non-coding sequence or fragment thereof (e.g., a promoter sequence, enhancer sequence, 5′ untranslated region (5′ UTR), 3′ untranslated region (3′ UTR), or a fragment thereof), a coding sequence of fragment thereof, an exon sequence or fragment thereof, an intron sequence or a fragment thereof.

### Target Capture Reagents

The methods described herein may comprise contacting a nucleic acid library with a plurality of target capture reagents in order to select and capture a plurality of specific target sequences (e.g., gene sequences or fragments thereof) for analysis. In some instances, a target capture reagent (i.e., a molecule which can bind to and thereby allow capture of a target molecule) is used to select the subject intervals to be analyzed. For example, a target capture reagent can be a bait molecule, e.g., a nucleic acid molecule (e.g., a DNA molecule or RNA molecule) which can hybridize to (i.e., is complementary to) a target molecule, and thereby allows capture of the target nucleic acid. In some instances, the target capture reagent, e.g., a bait molecule (or bait sequence), is a capture oligonucleotide (or capture probe). In some instances, the target nucleic acid is a genomic DNA molecule, an RNA molecule, a cDNA molecule derived from an RNA molecule, a microsatellite DNA sequence, and the like. In some instances, the target capture reagent is suitable for solution-phase hybridization to the target. In some instances, the target capture reagent is suitable for solid-phase hybridization to the target. In some instances, the target capture reagent is suitable for both solution-phase and solid-phase hybridization to the target. The design and construction of target capture reagents is described in more detail in, e.g., International Patent Application Publication No. WO 2020/236941, the entire content of which is incorporated herein by reference.

The methods described herein provide for optimized sequencing of a large number of genomic loci (e.g., genes or gene products (e.g., mRNA), microsatellite loci, etc.) from samples (e.g., cancerous tissue specimens, liquid biopsy samples, and the like) from one or more subjects by the appropriate selection of target capture reagents to select the target nucleic acid molecules to be sequenced. In some instances, a target capture reagent may hybridize to a specific target locus, e.g., a specific target gene locus or fragment thereof. In some instances, a target capture reagent may hybridize to a specific group of target loci, e.g., a specific group of gene loci or fragments thereof. In some instances, a plurality of target capture reagents comprising a mix of target-specific and/or group-specific target capture reagents may be used.

In some instances, the number of target capture reagents (e.g., bait molecules) in the plurality of target capture reagents (e.g., a bait set) contacted with a nucleic acid library to capture a plurality of target sequences for nucleic acid sequencing is greater than 10, greater than 50, greater than 100, greater than 200, greater than 300, greater than 400, greater than 500, greater than 600, greater than 700, greater than 800, greater than 900, greater than 1,000, greater than 1,250, greater than 1,500, greater than 1,750, greater than 2,000, greater than 3,000, greater than 4,000, greater than 5,000, greater than 10,000, greater than 25,000, or greater than 50,000.

In some instances, the overall length of the target capture reagent sequence can be between about 70 nucleotides and 1000 nucleotides. In one instance, the target capture reagent length is between about 100 and 300 nucleotides, 110 and 200 nucleotides, or 120 and 170 nucleotides, in length. In addition to those mentioned above, intermediate oligonucleotide lengths of about 70, 80, 90, 100, 110, 120, 130, 140, 150, 160, 170, 180, 190, 200, 210, 220, 230, 240, 250, 300, 400, 500, 600, 700, 800, and 900 nucleotides in length can be used in the methods described herein. In some embodiments, oligonucleotides of about 70, 80, 90, 100, 110, 120, 130, 140, 150, 160, 170, 180, 190, 200, 210, 220, or 230 bases can be used.

In some instances, each target capture reagent sequence can include: (i) a target-specific capture sequence (e.g., a gene locus or microsatellite locus-specific complementary sequence), (ii) an adapter, primer, barcode, and/or unique molecular identifier sequence, and (iii) universal tails on one or both ends. As used herein, the term “target capture reagent” can refer to the target-specific target capture sequence or to the entire target capture reagent oligonucleotide including the target-specific target capture sequence.

In some instances, the target-specific capture sequences in the target capture reagents are between about 40 nucleotides and 1000 nucleotides in length. In some instances, the target-specific capture sequence is between about 70 nucleotides and 300 nucleotides in length. In some instances, the target-specific sequence is between about 100 nucleotides and 200 nucleotides in length. In yet other instances, the target-specific sequence is between about 120 nucleotides and 170 nucleotides in length, typically 120 nucleotides in length. Intermediate lengths in addition to those mentioned above also can be used in the methods described herein, such as target-specific sequences of about 40, 50, 60, 70, 80, 90, 100, 110, 120, 130, 140, 150, 160, 170, 180, 190, 200, 210, 220, 230, 240, 250, 300, 400, 500, 600, 700, 800, and 900 nucleotides in length, as well as target-specific sequences of lengths between the above-mentioned lengths.

In some instances, the target capture reagent may be designed to select a subject interval containing one or more rearrangements, e.g., an intron containing a genomic rearrangement. In such instances, the target capture reagent is designed such that repetitive sequences are masked to increase the selection efficiency. In those instances where the rearrangement has a known juncture sequence, complementary target capture reagents can be designed to recognize the juncture sequence to increase the selection efficiency.

In some instances, the disclosed methods may comprise the use of target capture reagents designed to capture two or more different target categories, each category having a different target capture reagent design strategy. In some instances, the hybridization-based capture methods and target capture reagent compositions disclosed herein may provide for the capture and homogeneous coverage of a set of target sequences, while minimizing coverage of genomic sequences outside of the targeted set of sequences. In some instances, the target sequences may include the entire exome of genomic DNA or a selected subset thereof. In some instances, the target sequences may include, e.g., a large chromosomal region (e.g., a whole chromosome arm). The methods and compositions disclosed herein provide different target capture reagents for achieving different sequencing depths and patterns of coverage for complex sets of target nucleic acid sequences.

Typically, DNA molecules are used as target capture reagent sequences, although RNA molecules can also be used. In some instances, a DNA molecule target capture reagent can be single stranded DNA (ssDNA) or double-stranded DNA (dsDNA). In some instances, an RNA-DNA duplex is more stable than a DNA-DNA duplex and therefore provides for potentially better capture of nucleic acids.

In some instances, the disclosed methods comprise providing a selected set of nucleic acid molecules (e.g., a library catch) captured from one or more nucleic acid libraries. For example, the method may comprise: providing one or a plurality of nucleic acid libraries, each comprising a plurality of nucleic acid molecules (e.g., a plurality of target nucleic acid molecules and/or reference nucleic acid molecules) extracted from one or more samples from one or more subjects; contacting the one or a plurality of libraries (e.g., in a solution-based hybridization reaction) with one, two, three, four, five, or more than five pluralities of target capture reagents (e.g., oligonucleotide target capture reagents) to form a hybridization mixture comprising a plurality of target capture reagent/nucleic acid molecule hybrids; separating the plurality of target capture reagent/nucleic acid molecule hybrids from said hybridization mixture, e.g., by contacting said hybridization mixture with a binding entity that allows for separation of said plurality of target capture reagent/nucleic acid molecule hybrids from the hybridization mixture, thereby providing a library catch (e.g., a selected or enriched subgroup of nucleic acid molecules from the one or a plurality of libraries).

In some instances, the disclosed methods may further comprise amplifying the library catch (e.g., by performing PCR). In other instances, the library catch is not amplified.

In some instances, the target capture reagents can be part of a kit which can optionally comprise instructions, standards, buffers or enzymes or other reagents.

### Hybridization Conditions

As noted above, the methods disclosed herein may include the step of contacting the library (e.g., the nucleic acid library) with a plurality of target capture reagents to provide a selected library target nucleic acid sequences (i.e., the library catch). The contacting step can be effected in, e.g., solution-based hybridization. In some instances, the method includes repeating the hybridization step for one or more additional rounds of solution-based hybridization. In some instances, the method further includes subjecting the library catch to one or more additional rounds of solution-based hybridization with the same or a different collection of target capture reagents.

In some instances, the contacting step is effected using a solid support, e.g., an array. Suitable solid supports for hybridization are described in, e.g., Albert, T. J. et al. (2007) Nat. Methods 4 (11): 903-5; Hodges, E. et al. (2007) Nat. Genet. 39 (12): 1522-7; and Okou, D. T. et al. (2007) Nat. Methods 4 (11): 907-9, the contents of which are incorporated herein by reference in their entireties.

Hybridization methods that can be adapted for use in the methods herein are described in the art, e.g., as described in International Patent Application Publication No. WO 2012/092426. Methods for hybridizing target capture reagents to a plurality of target nucleic acids are described in more detail in, e.g., International Patent Application Publication No. WO 2020/236941, the entire content of which is incorporated herein by reference.

### Sequencing Methods

The methods and systems disclosed herein can be used in combination with, or as part of, a method or system for sequencing nucleic acids (e.g., a next-generation sequencing system) to generate a plurality of sequence reads that overlap one or more gene loci within a subgenomic interval in the sample and thereby determine, e.g., gene allele sequences at a plurality of gene loci. “Next-generation sequencing” (or “NGS”) as used herein may also be referred to as “massively parallel sequencing”, and refers to any sequencing method that determines the nucleotide sequence of either individual nucleic acid molecules (e.g., as in single molecule sequencing) or clonally expanded proxies for individual nucleic acid molecules in a high throughput fashion (e.g., wherein greater than 10³, 10⁴, 10⁵ or more than 10⁵ molecules are sequenced simultaneously).

Next-generation sequencing methods are known in the art, and are described in, e.g., Metzker, M. (2010) Nature Biotechnology Reviews 11:31-46, which is incorporated herein by reference. Other examples of sequencing methods suitable for use when implementing the methods and systems disclosed herein are described in, e.g., International Patent Application Publication No. WO 2012/092426. In some instances, the sequencing may comprise, for example, whole genome sequencing (WGS), whole exome sequencing, targeted sequencing, or direct sequencing. In some instances, sequencing may be performed using, e.g., Sanger sequencing. In some instances, the sequencing may comprise a paired-end sequencing technique that allows both ends of a fragment to be sequenced and generates high-quality, alignable sequence data for detection of, e.g., genomic rearrangements, repetitive sequence elements, gene fusions, and novel transcripts.

The disclosed methods and systems may be implemented using sequencing platforms such as the Roche 454, Illumina Solexa, ABI-SOLID, ION Torrent, Complete Genomics, Pacific Bioscience, Helicos, and/or the Polonator platform. In some instances, sequencing may comprise Illumina MiSeq sequencing. In some instances, sequencing may comprise Illumina HiSeq sequencing. In some instances, sequencing may comprise Illumina NovaSeq sequencing. Optimized methods for sequencing a large number of target genomic loci in nucleic acids extracted from a sample are described in more detail in, e.g., International Patent Application Publication No. WO 2020/236941, the entire content of which is incorporated herein by reference.

In certain instances, the disclosed methods comprise one or more of the steps of: (a) acquiring a library comprising a plurality of normal and/or tumor nucleic acid molecules from a sample; (b) simultaneously or sequentially contacting the library with one, two, three, four, five, or more than five pluralities of target capture reagents under conditions that allow hybridization of the target capture reagents to the target nucleic acid molecules, thereby providing a selected set of captured normal and/or tumor nucleic acid molecules (i.e., a library catch); (c) separating the selected subset of the nucleic acid molecules (e.g., the library catch) from the hybridization mixture, e.g., by contacting the hybridization mixture with a binding entity that allows for separation of the target capture reagent/nucleic acid molecule hybrids from the hybridization mixture, (d) sequencing the library catch to acquiring a plurality of reads (e.g., sequence reads) that overlap one or more subject intervals (e.g., one or more target sequences) from said library catch that may comprise a mutation (or alteration), e.g., a variant sequence comprising a somatic mutation or germline mutation; (e) aligning said sequence reads using an alignment method as described elsewhere herein; and/or (f) assigning a nucleotide value for a nucleotide position in the subject interval (e.g., calling a mutation using, e.g., a Bayesian method or other method described herein) from one or more sequence reads of the plurality.

In some instances, acquiring sequence reads for one or more subject intervals may comprise sequencing at least 1, at least 5, at least 10, at least 20, at least 30, at least 40, at least 50, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1,000, at least 1,250, at least 1,500, at least 1,750, at least 2,000, at least 2,250, at least 2,500, at least 2,750, at least 3,000, at least 3,500, at least 4,000, at least 4,500, or at least 5,000 loci, e.g., genomic loci, gene loci, microsatellite loci, etc. In some instances, acquiring a sequence read for one or more subject intervals may comprise sequencing a subject interval for any number of loci within the range described in this paragraph, e.g., for at least 2,850 gene loci.

In some instances, acquiring a sequence read for one or more subject intervals comprises sequencing a subject interval with a sequencing method that provides a sequence read length (or average sequence read length) of at least 20 bases, at least 30 bases, at least 40 bases, at least 50 bases, at least 60 bases, at least 70 bases, at least 80 bases, at least 90 bases, at least 100 bases, at least 120 bases, at least 140 bases, at least 160 bases, at least 180 bases, at least 200 bases, at least 220 bases, at least 240 bases, at least 260 bases, at least 280 bases, at least 300 bases, at least 320 bases, at least 340 bases, at least 360 bases, at least 380 bases, or at least 400 bases. In some instances, acquiring a sequence read for the one or more subject intervals may comprise sequencing a subject interval with a sequencing method that provides a sequence read length (or average sequence read length) of any number of bases within the range described in this paragraph, e.g., a sequence read length (or average sequence read length) of 56 bases.

In some instances, acquiring a sequence read for one or more subject intervals may comprise sequencing with at least 100× or more coverage (or depth) on average. In some instances, acquiring a sequence read for one or more subject intervals may comprise sequencing with at least 100×, at least 150×, at least 200×, at least 250×, at least 500×, at least 750×, at least 1,000×, at least 1,500×, at least 2,000×, at least 2,500×, at least 3,000×, at least 3,500×, at least 4,000×, at least 4,500×, at least 5,000×, at least 5,500×, or at least 6,000× or more coverage (or depth) on average. In some instances, acquiring a sequence read for one or more subject intervals may comprise sequencing with an average coverage (or depth) having any value within the range of values described in this paragraph, e.g., at least 160×.

In some instances, acquiring a read for the one or more subject intervals comprises sequencing with an average sequencing depth having any value ranging from at least 100× to at least 6,000× for greater than about 90%, 92%, 94%, 95%, 96%, 97%, 98%, or 99% of the gene loci sequenced. For example, in some instances acquiring a read for the subject interval comprises sequencing with an average sequencing depth of at least 125× for at least 99% of the gene loci sequenced. As another example, in some instances acquiring a read for the subject interval comprises sequencing with an average sequencing depth of at least 4,100× for at least 95% of the gene loci sequenced.

In some instances, the relative abundance of a nucleic acid species in the library can be estimated by counting the relative number of occurrences of their cognate sequences (e.g., the number of sequence reads for a given cognate sequence) in the data generated by the sequencing experiment.

In some instances, the disclosed methods and systems provide nucleotide sequences for a set of subject intervals (e.g., gene loci), as described herein. In certain instances, the sequences are provided without using a method that includes a matched normal control (e.g., a wild-type control) and/or a matched tumor control (e.g., primary versus metastatic).

In some instances, the level of sequencing depth as used herein (e.g., an X-fold level of sequencing depth) refers to the number of reads (e.g., unique reads) obtained after detection and removal of duplicate reads (e.g., PCR duplicate reads). In other instances, duplicate reads are evaluated, e.g., to support detection of copy number alteration (CNAs).

### Alignment

Alignment is the process of matching a read with a location, e.g., a genomic location or locus. In some instances, NGS reads may be aligned to a known reference sequence (e.g., a wild-type sequence). In some instances, NGS reads may be assembled de novo. Methods of sequence alignment for NGS reads are described in, e.g., Trapnell, C. and Salzberg, S. L. Nature Biotech., 2009, 27:455-457. Examples of de novo sequence assemblies are described in, e.g., Warren R., et al., Bioinformatics, 2007, 23:500-501; Butler, J. et al., Genome Res., 2008, 18:810-820; and Zerbino, D. R. and Birney, E., Genome Res., 2008, 18:821-829. Optimization of sequence alignment is described in the art, e.g., as set out in International Patent Application Publication No. WO 2012/092426. Additional description of sequence alignment methods is provided in, e.g., International Patent Application Publication No. WO 2020/236941, the entire content of which is incorporated herein by reference.

Misalignment (e.g., the placement of base-pairs from a short read at incorrect locations in the genome), e.g., misalignment of reads due to sequence context (e.g., the presence of repetitive sequence) around an actual cancer mutation can lead to reduction in sensitivity of mutation detection, can lead to a reduction in sensitivity of mutation detection, as reads for the alternate allele may be shifted off the histogram peak of alternate allele reads. Other examples of sequence context that may cause misalignment include short-tandem repeats, interspersed repeats, low complexity regions, insertions-deletions (indels), and paralogs. If the problematic sequence context occurs where no actual mutation is present, misalignment may introduce artifactual reads of “mutated” alleles by placing reads of actual reference genome base sequences at the wrong location. Because mutation-calling algorithms for multigene analysis should be sensitive to even low-abundance mutations, sequence misalignments may increase false positive discovery rates and/or reduce specificity.

In some instances, the methods and systems disclosed herein may integrate the use of multiple, individually-tuned, alignment methods or algorithms to optimize base-calling performance in sequencing methods, particularly in methods that rely on massively parallel sequencing of a large number of diverse genetic events at a large number of diverse genomic loci. In some instances, the disclosed methods and systems may comprise the use of one or more global alignment algorithms. In some instances, the disclosed methods and systems may comprise the use of one or more local alignment algorithms. Examples of alignment algorithms that may be used include, but are not limited to, the Burrows-Wheeler Alignment (BWA) software bundle (see, e.g., Li, et al. (2009), “Fast and Accurate Short Read Alignment with Burrows-Wheeler Transform”, Bioinformatics 25:1754-60; Li, et al. (2010), Fast and Accurate Long-Read Alignment with Burrows-Wheeler Transform”, Bioinformatics epub. PMID: 20080505), the Smith-Waterman algorithm (see, e.g., Smith, et al. (1981), “Identification of Common Molecular Subsequences”, J. Molecular Biology 147 (1): 195-197), the Striped Smith-Waterman algorithm (see, e.g., Farrar (2007), “Striped Smith-Waterman Speeds Database Searches Six Times Over Other SIMD Implementations”, Bioinformatics 23 (2): 156-161), the Needleman-Wunsch algorithm (Needleman, et al. (1970) “A General Method Applicable to the Search for Similarities in the Amino Acid Sequence of Two Proteins”, J. Molecular Biology 48 (3): 443-53), or any combination thereof.

In some instances, the methods and systems disclosed herein may also comprise the use of a sequence assembly algorithm, e.g., the Arachne sequence assembly algorithm (see, e.g., Batzoglou, et al. (2002), “ARACHNE: A Whole-Genome Shotgun Assembler”, Genome Res. 12:177-189).

In some instances, the alignment method used to analyze sequence reads is not individually customized or tuned for detection of different variants (e.g., point mutations, insertions, deletions, and the like) at different genomic loci. In some instances, different alignment methods are used to analyze reads that are individually customized or tuned for detection of at least a subset of the different variants detected at different genomic loci. In some instances, different alignment methods are used to analyze reads that are individually customized or tuned to detect each different variant at different genomic loci. In some instances, tuning can be a function of one or more of: (i) the genetic locus (e.g., gene loci, microsatellite locus, or other subject interval) being sequenced, (ii) the tumor type associated with the sample, (iii) the variant being sequenced, or (iv) a characteristic of the sample or the subject. The selection or use of alignment conditions that are individually tuned to a number of specific subject intervals to be sequenced allows optimization of speed, sensitivity, and specificity. The method is particularly effective when the alignment of reads for a relatively large number of diverse subject intervals are optimized. In some instances, the method includes the use of an alignment method optimized for rearrangements in combination with other alignment methods optimized for subject intervals not associated with rearrangements.

In some instances, the methods disclosed herein further comprise selecting or using an alignment method for analyzing, e.g., aligning, a sequence read, wherein said alignment method is a function of, is selected responsive to, or is optimized for, one or more of: (i) tumor type, e.g., the tumor type in the sample; (ii) the location (e.g., a gene locus) of the subject interval being sequenced; (iii) the type of variant (e.g., a point mutation, insertion, deletion, substitution, copy number variation (CNV), rearrangement, or fusion) in the subject interval being sequenced; (iv) the site (e.g., nucleotide position) being analyzed; (v) the type of sample (e.g., a sample described herein); and/or (vi) adjacent sequence(s) in or near the subject interval being evaluated (e.g., according to the expected propensity thereof for misalignment of the subject interval due to, e.g., the presence of repeated sequences in or near the subject interval).

In some instances, the methods disclosed herein allow for the rapid and efficient alignment of troublesome reads, e.g., a read having a rearrangement. Thus, in some instances where a read for a subject interval comprises a nucleotide position with a rearrangement, e.g., a translocation, the method can comprise using an alignment method that is appropriately tuned and that includes: (i) selecting a rearrangement reference sequence for alignment with a read, wherein said rearrangement reference sequence aligns with a rearrangement (in some instances, the reference sequence is not identical to the genomic rearrangement); and (ii) comparing, e.g., aligning, a read with said rearrangement reference sequence.

In some instances, alternative methods may be used to align troublesome reads. These methods are particularly effective when the alignment of reads for a relatively large number of diverse subject intervals is optimized. By way of example, a method of analyzing a sample can comprise: (i) performing a comparison (e.g., an alignment comparison) of a read using a first set of parameters (e.g., using a first mapping algorithm, or by comparison with a first reference sequence), and determining if said read meets a first alignment criterion (e.g., the read can be aligned with said first reference sequence, e.g., with less than a specific number of mismatches); (ii) if said read fails to meet the first alignment criterion, performing a second alignment comparison using a second set of parameters, (e.g., using a second mapping algorithm, or by comparison with a second reference sequence); and (iii) optionally, determining if said read meets said second criterion (e.g., the read can be aligned with said second reference sequence, e.g., with less than a specific number of mismatches), wherein said second set of parameters comprises use of, e.g., said second reference sequence, which, compared with said first set of parameters, is more likely to result in an alignment with a read for a variant (e.g., a rearrangement, insertion, deletion, or translocation).

In some instances, the alignment of sequence reads in the disclosed methods may be combined with a mutation calling method as described elsewhere herein. As discussed herein, reduced sensitivity for detecting actual mutations may be addressed by evaluating the quality of alignments (manually or in an automated fashion) around expected mutation sites in the genes or genomic loci (e.g., gene loci) being analyzed. In some instances, the sites to be evaluated can be obtained from databases of the human genome (e.g., the HG19 human reference genome) or cancer mutations (e.g., COSMIC). Regions that are identified as problematic can be remedied with the use of an algorithm selected to give better performance in the relevant sequence context, e.g., by alignment optimization (or re-alignment) using slower, but more accurate alignment algorithms such as Smith-Waterman alignment. In cases where general alignment algorithms cannot remedy the problem, customized alignment approaches may be created by, e.g., adjustment of maximum difference mismatch penalty parameters for genes with a high likelihood of containing substitutions; adjusting specific mismatch penalty parameters based on specific mutation types that are common in certain tumor types (e.g. C→T in melanoma); or adjusting specific mismatch penalty parameters based on specific mutation types that are common in certain sample types (e.g. substitutions that are common in FFPE).

Reduced specificity (increased false positive rate) in the evaluated subject intervals due to misalignment can be assessed by manual or automated examination of all mutation calls in the sequencing data. Those regions found to be prone to spurious mutation calls due to misalignment can be subjected to alignment remedies as discussed above. In cases where no algorithmic remedy is found possible, “mutations” from the problem regions can be classified or screened out from the panel of targeted loci.

### Mutation Calling

Base calling refers to the raw output of a sequencing device, e.g., the determined sequence of nucleotides in an oligonucleotide molecule. Mutation calling refers to the process of selecting a nucleotide value, e.g., A, G, T, or C, for a given nucleotide position being sequenced. Typically, the sequence reads (or base calling) for a position will provide more than one value, e.g., some reads will indicate a T and some will indicate a G. Mutation calling is the process of assigning a correct nucleotide value, e.g., one of those values, to the sequence. Although it is referred to as “mutation” calling, it can be applied to assign a nucleotide value to any nucleotide position, e.g., positions corresponding to mutant alleles, wild-type alleles, alleles that have not been characterized as either mutant or wild-type, or to positions not characterized by variability.

In some instances, the disclosed methods may comprise the use of customized or tuned mutation calling algorithms or parameters thereof to optimize performance when applied to sequencing data, particularly in methods that rely on massively parallel sequencing of a large number of diverse genetic events at a large number of diverse genomic loci (e.g., gene loci, microsatellite regions, etc.) in samples, e.g., samples from a subject having cancer. Optimization of mutation calling is described in the art, e.g., as set out in International Patent Application Publication No. WO 2012/092426.

Methods for mutation calling can include one or more of the following: making independent calls based on the information at each position in the reference sequence (e.g., examining the sequence reads; examining the base calls and quality scores; calculating the probability of observed bases and quality scores given a potential genotype; and assigning genotypes (e.g., using Bayes' rule)); removing false positives (e.g., using depth thresholds to reject SNPs with read depth much lower or higher than expected; local realignment to remove false positives due to small indels); and performing linkage disequilibrium (LD)/imputation-based analysis to refine the calls.

Equations used to calculate the genotype likelihood associated with a specific genotype and position are described in, e.g., Li, H. and Durbin, R. Bioinformatics, 2010; 26 (5): 589-95. The prior expectation for a particular mutation in a certain cancer type can be used when evaluating samples from that cancer type. Such likelihood can be derived from public databases of cancer mutations, e.g., Catalogue of Somatic Mutation in Cancer (COSMIC), HGMD (Human Gene Mutation Database), The SNP Consortium, Breast Cancer Mutation Data Base (BIC), and Breast Cancer Gene Database (BCGD).

Examples of LD/imputation based analysis are described in, e.g., Browning, B. L. and Yu, Z. Am. J. Hum. Genet. 2009, 85 (6): 847-61. Examples of low-coverage SNP calling methods are described in, e.g., Li, Y., et al., Annu. Rev. Genomics Hum. Genet. 2009, 10:387-406.

After alignment, detection of substitutions can be performed using a mutation calling method (e.g., a Bayesian mutation calling method) which is applied to each base in each of the subject intervals, e.g., exons of a gene or other locus to be evaluated, where presence of alternate alleles is observed. This method will compare the probability of observing the read data in the presence of a mutation with the probability of observing the read data in the presence of base-calling error alone. Mutations can be called if this comparison is sufficiently strongly supportive of the presence of a mutation.

An advantage of a Bayesian mutation-detection approach is that the comparison of the probability of the presence of a mutation with the probability of base-calling error alone can be weighted by a prior expectation of the presence of a mutation at the site. If some reads of an alternate allele are observed at a frequently mutated site for the given cancer type, then presence of a mutation may be confidently called even if the amount of evidence of mutation does not meet the usual thresholds. This flexibility can then be used to increase detection sensitivity for even rarer mutations/lower purity samples, or to make the test more robust to decreases in read coverage. The likelihood of a random base-pair in the genome being mutated in cancer is ˜1e−6. The likelihood of specific mutations occurring at many sites in, for example, a typical multigenic cancer genome panel can be orders of magnitude higher. These likelihoods can be derived from public databases of cancer mutations (e.g., COSMIC).

Indel calling is a process of finding bases in the sequencing data that differ from the reference sequence by insertion or deletion, typically including an associated confidence score or statistical evidence metric. Methods of indel calling can include the steps of identifying candidate indels, calculating genotype likelihood through local re-alignment, and performing LD-based genotype inference and calling. Typically, a Bayesian approach is used to obtain potential indel candidates, and then these candidates are tested together with the reference sequence in a Bayesian framework.

Algorithms to generate candidate indels are described in, e.g., McKenna, A., et al., Genome Res. 2010; 20 (9):1297-303; Ye, K., et al., Bioinformatics, 2009; 25 (21):2865-71; Lunter, G., and Goodson, M., Genome Res. 2011; 21 (6):936-9; and Li, H., et al. (2009), Bioinformatics 25 (16): 2078-9.

Methods for generating indel calls and individual-level genotype likelihoods include, e.g., the Dindel algorithm (Albers, C. A., et al., Genome Res. 2011; 21 (6):961-73). For example, the Bayesian EM algorithm can be used to analyze the reads, make initial indel calls, and generate genotype likelihoods for each candidate indel, followed by imputation of genotypes using, e.g., QCALL (Le S. Q. and Durbin R. Genome Res. 2011; 21 (6):952-60). Parameters, such as prior expectations of observing the indel can be adjusted (e.g., increased or decreased), based on the size or location of the indels.

Methods have been developed that address limited deviations from allele frequencies of 50% or 100% for the analysis of cancer DNA. (see, e.g., SNVMix-Bioinformatics. 2010 Mar. 15; 26 (6): 730-736.) Methods disclosed herein, however, allow consideration of the possibility of the presence of a mutant allele at frequencies (or allele fractions) ranging from 1% to 100% (i.e., allele fractions ranging from 0.01 to 1.0), and especially at levels lower than 50%. This approach is particularly important for the detection of mutations in, for example, low-purity FFPE samples of natural (multi-clonal) tumor DNA.

In some instances, the mutation calling method used to analyze sequence reads is not individually customized or fine-tuned for detection of different mutations at different genomic loci. In some instances, different mutation calling methods are used that are individually customized or fine-tuned for at least a subset of the different mutations detected at different genomic loci. In some instances, different mutation calling methods are used that are individually customized or fine-tuned for each different mutant detected at each different genomic loci. The customization or tuning can be based on one or more of the factors described herein, e.g., the type of cancer in a sample, the gene or locus in which the subject interval to be sequenced is located, or the variant to be sequenced. This selection or use of mutation calling methods individually customized or fine-tuned for a number of subject intervals to be sequenced allows for optimization of speed, sensitivity and specificity of mutation calling.

In some instances, a nucleotide value is assigned for a nucleotide position in each of X unique subject intervals using a unique mutation calling method, and X is at least 2, at least 3, at least 4, at least 5, at least 10, at least 15, at least 20, at least 30, at least 40, at least 50, at least 60, at least 70, at least 80, at least 90, at least 100, at least 200, at least 300, at least 400, at least 500, at least 1000, at least 1500, at least 2000, at least 2500, at least 3000, at least 3500, at least 4000, at least 4500, at least 5000, or greater. The calling methods can differ, and thereby be unique, e.g., by relying on different Bayesian prior values.

In some instances, assigning said nucleotide value is a function of a value which is or represents the prior (e.g., literature) expectation of observing a read showing a variant, e.g., a mutation, at said nucleotide position in a tumor of type.

In some instances, the method comprises assigning a nucleotide value (e.g., calling a mutation) for at least 10, 20, 40, 50, 60, 70, 80, 90, 100, 200, 300, 400, 500, 600, 700, 800, 900, or 1,000 nucleotide positions, wherein each assignment is a function of a unique value (as opposed to the value for the other assignments) which is or represents the prior (e.g., literature) expectation of observing a read showing a variant, e.g., a mutation, at said nucleotide position in a tumor of type.

In some instances, assigning said nucleotide value is a function of a set of values which represent the probabilities of observing a read showing said variant at said nucleotide position if the variant is present in the sample at a specified frequency (e.g., 1%, 5%, 10%, etc.) and/or if the variant is absent (e.g., observed in the reads due to base-calling error alone).

In some instances, the mutation calling methods described herein can include the following: (a) acquiring, for a nucleotide position in each of said X subject intervals: (i) a first value which is or represents the prior (e.g., literature) expectation of observing a read showing a variant, e.g., a mutation, at said nucleotide position in a tumor of type X; and (ii) a second set of values which represent the probabilities of observing a read showing said variant at said nucleotide position if the variant is present in the sample at a frequency (e.g., 1%, 5%, 10%, etc.) and/or if the variant is absent (e.g., observed in the reads due to base-calling error alone); and (b) responsive to said values, assigning a nucleotide value (e.g., calling a mutation) from said reads for each of said nucleotide positions by weighing, e.g., by a Bayesian method described herein, the comparison among the values in the second set using the first value (e.g., computing the posterior probability of the presence of a mutation), thereby analyzing said sample.

Additional description of mutation calling methods is provided in, e.g., International Patent Application Publication No. WO 2020/236941, the entire content of which is incorporated herein by reference.

### Systems

Also disclosed herein are systems designed to implement any of the disclosed methods for predicting the likelihood of success for performing GP analysis on a sample from a subject. The systems may comprise, e.g., one or more processors, and a memory unit communicatively coupled to the one or more processors and configured to store instructions that, when executed by the one or more processors, cause the system to: receive the data for a plurality of pre-analytical variables associated with a sample derived from a subject; apply the received data to a multivariable model trained to predict outcomes for GP assays; generate a prediction of the likelihood of success for performing GP of the sample based on the applied multivariable model; and report the prediction of the likelihood of success for performing GP of the sample.

In some instances, the disclosed systems may further comprise a sequencer, e.g., a next generation sequencer (also referred to as a massively parallel sequencer). Examples of next generation (or massively parallel) sequencing platforms include, but are not limited to, the Roche 454, Illumina Solexa, ABI-SOLID, ION Torrent, or Pacific Bioscience sequencing platforms.

In some instances, the disclosed systems may be used for predicting the likelihood of success for performing GP analysis in any of a variety of samples as described herein (e.g., a tissue sample, biopsy sample, hematological sample, or liquid biopsy sample derived from the subject).

In some instance, the nucleic acid sequence data is acquired using a next generation sequencing technique (also referred to as a massively parallel sequencing technique) having a read-length of less than 400 bases, less than 300 bases, less than 200 bases, less than 150 bases, less than 100 bases, less than 90 bases, less than 80 bases, less than 70 bases, less than 60 bases, less than 50 bases, less than 40 bases, or less than 30 bases.

In some instances, the determination of a successful GP assay result may be used to select, initiate, adjust, or terminate a treatment for cancer in the subject (e.g., a patient) from which the sample was derived, as described elsewhere herein.

In some instances, the disclosed systems may further comprise sample processing and library preparation workstations, microplate-handling robotics, fluid dispensing systems, temperature control modules, environmental control chambers, additional data storage modules, data communication modules (e.g., Bluetooth®, WiFi, intranet, or internet communication hardware and associated software), display modules, one or more local and/or cloud-based software packages (e.g., instrument/system control software packages, sequencing data analysis software packages), etc., or any combination thereof. In some instances, the systems may comprise, or be part of, a computer system or computer network as described elsewhere herein.

### Computer Systems and Networks

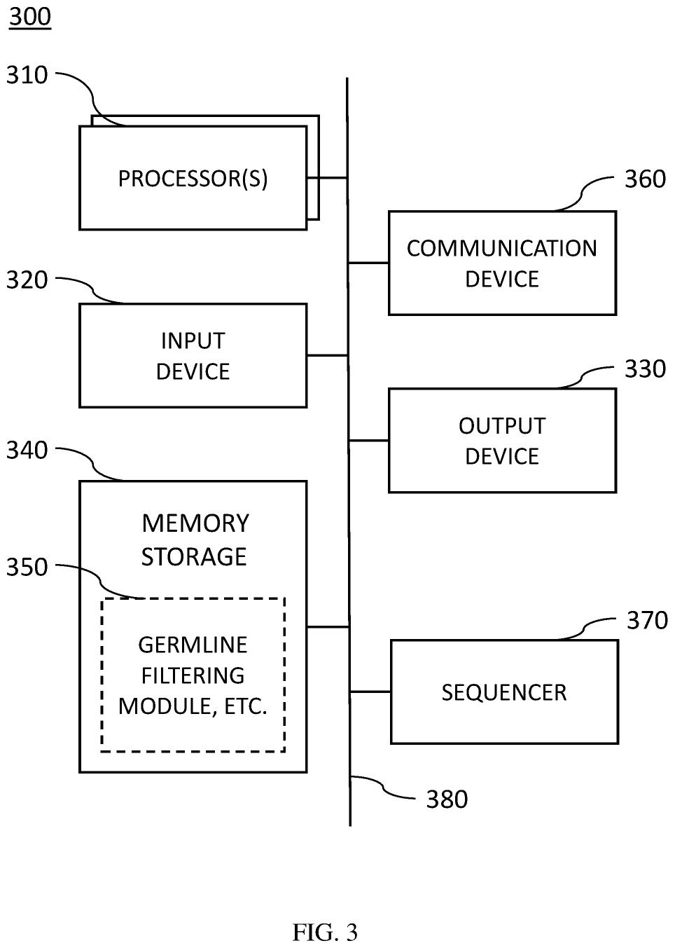

FIG. 3 illustrates an example of a computing device or system in accordance with one embodiment. Device 300 can be a host computer connected to a network. Device 300 can be a client computer or a server. As shown in FIG. 3, device 300 can be any suitable type of microprocessor-based device, such as a personal computer, workstation, server or handheld computing device (portable electronic device) such as a phone or tablet. The device can include, for example, one or more processor(s) 310, input devices 320, output devices 330, memory or storage devices 340, communication devices 360, and nucleic acid sequencers 370. Software 350 residing in memory or storage device 340 may comprise, e.g., an operating system as well as software for executing the methods described herein. Input device 320 and output device 330 can generally correspond to those described herein, and can either be connectable or integrated with the computer.

Input device 320 can be any suitable device that provides input, such as a touch screen, keyboard or keypad, mouse, or voice-recognition device. Output device 330 can be any suitable device that provides output, such as a touch screen, haptics device, or speaker.

Storage 340 can be any suitable device that provides storage (e.g., an electrical, magnetic or optical memory including a RAM (volatile and non-volatile), cache, hard drive, or removable storage disk). Communication device 360 can include any suitable device capable of transmitting and receiving signals over a network, such as a network interface chip or device. The components of the computer can be connected in any suitable manner, such as via a wired media (e.g., a physical system bus 380, Ethernet connection, or any other wire transfer technology) or wirelessly (e.g., Bluetooth®, Wi-Fi®, or any other wireless technology).

Software module 350, which can be stored as executable instructions in storage 340 and executed by processor(s) 310, can include, for example, an operating system and/or the processes that embody the functionality of the methods of the present disclosure (e.g., as embodied in the devices as described herein).

Software module 350 can also be stored and/or transported within any non-transitory computer-readable storage medium for use by or in connection with an instruction execution system, apparatus, or device, such as those described herein, that can fetch instructions associated with the software from the instruction execution system, apparatus, or device and execute the instructions. In the context of this disclosure, a computer-readable storage medium can be any medium, such as storage 340, that can contain or store processes for use by or in connection with an instruction execution system, apparatus, or device. Examples of computer-readable storage media may include memory units like hard drives, flash drives and distribute modules that operate as a single functional unit. Also, various processes described herein may be embodied as modules configured to operate in accordance with the embodiments and techniques described above. Further, while processes may be shown and/or described separately, those skilled in the art will appreciate that the above processes may be routines or modules within other processes.

Software module 350 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

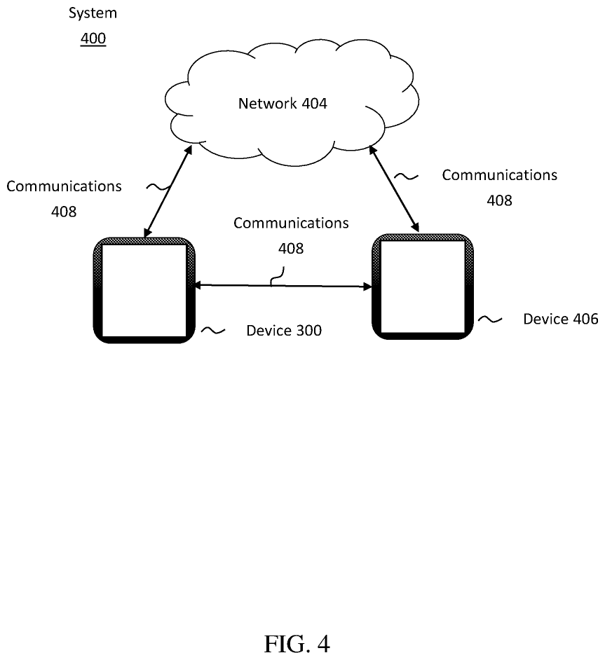

Device 300 may be connected to a network (e.g., network 404, as shown in FIG. 4 and/or described below), which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 300 can be implemented using any operating system, e.g., an operating system suitable for operating on the network. Software module 350 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example. In some embodiments, the operating system is executed by one or more processors, e.g., processor(s) 310.

Device 300 can further include a sequencer 370, which can be any suitable nucleic acid sequencing instrument.

FIG. 4 illustrates an example of a computing system in accordance with one embodiment. In system 400, device 300 (e.g., as described above and illustrated in FIG. 3) is connected to network 404, which is also connected to device 406. In some embodiments, device 406 is a sequencer. Exemplary sequencers can include, without limitation, Roche/454's Genome Sequencer (GS) FLX System, Illumina/Solexa's Genome Analyzer (GA), Illumina's HiSeq 2500, HiSeq 3000, HiSeq 4000 and NovaSeq 6000 Sequencing Systems, Life/APG's Support Oligonucleotide Ligation Detection (SOLID) system, Polonator's G.007 system, Helicos BioSciences' HeliScope Gene Sequencing system, or Pacific Biosciences' PacBio RS system.

Devices 300 and 406 may communicate, e.g., using suitable communication interfaces via network 404, such as a Local Area Network (LAN), Virtual Private Network (VPN), or the Internet. In some embodiments, network 404 can be, for example, the Internet, an intranet, a virtual private network, a cloud network, a wired network, or a wireless network. Devices 300 and 406 may communicate, in part or in whole, via wireless or hardwired communications, such as Ethernet, IEEE 802.11b wireless, or the like. Additionally, devices 300 and 406 may communicate, e.g., using suitable communication interfaces, via a second network, such as a mobile/cellular network. Communication between devices 300 and 406 may further include or communicate with various servers such as a mail server, mobile server, media server, telephone server, and the like. In some embodiments, Devices 300 and 406 can communicate directly (instead of, or in addition to, communicating via network 404), e.g., via wireless or hardwired communications, such as Ethernet, IEEE 802.11b wireless, or the like. In some embodiments, devices 300 and 406 communicate via communications 408, which can be a direct connection or can occur via a network (e.g., network 404).

One or all of devices 300 and 406 generally include logic (e.g., http web server logic) or are programmed to format data, accessed from local or remote databases or other sources of data and content, for providing and/or receiving information via network 404 according to various examples described herein.

### EXEMPLARY IMPLEMENTATIONS

Exemplary implementations of the methods and systems described herein include:

1. A method for predicting a likelihood of success for performing genomic profiling of a sample derived from a subject, comprising:
receiving, using one or more processors, data for a plurality of pre-analytical variables associated with the sample;
applying, using the one or more processors, the received data to a multivariable model trained to predict outcomes for genomic profiling assays;
generating, using the one or more processors, a prediction of the likelihood of success for performing genomic profiling of the sample based on the applied multivariable model; and
reporting, using the one or more processors, the prediction of the likelihood of success for performing genomic profiling of the sample.

2. The method of clause 1, further comprising:
based on the prediction of the likelihood of success being equal to or greater than a predefined threshold, providing a plurality of nucleic acid molecules obtained from the sample from the subject;
ligating one or more adapters onto one or more nucleic acid molecules from the plurality of nucleic acid molecules;
amplifying the one or more ligated nucleic acid molecules from the plurality of nucleic acid molecules;
capturing amplified nucleic acid molecules from the amplified nucleic acid molecules;
sequencing, by a sequencer, the captured nucleic acid molecules to obtain a plurality of sequence reads that represent the captured nucleic acid molecules and that overlap one or more gene loci within one or more subgenomic intervals in the sample; and
generating, by one or more processors, a genomic profile including sequence read analysis data based on the sequence reads for the sample.

3. The method of clause 1 or clause 2, further comprising:
training, by the one or more processors, the multivariable model using training data.

4. The method of clause 3, wherein the training data comprises data derived from univariate analyses of clinical study data for samples collected from subjects representing a range of subject age, subject sex, diagnosis, stage of disease, sample type, sample collection site, sample collection method, sample preparation method, sample preservation method, sample age, sample transportation method, or any combination thereof.

5. The method of clause 4, wherein the training data further comprises data for ECOG status, subject treatment status, tumor cellularity in the sample, tumor nuclei content in the sample, tissue surface area of the sample, tissue matrix in the sample, prior genomic profiling assay results, or any combination thereof.

6. The method of any one of clauses 1 to 5, wherein the plurality of pre-analytical variables comprises subject age, subject sex, diagnosis, stage of disease, sample type, sample collection site, sample collection method, sample preparation method, sample preservation method, sample age, sample transportation method, or any combination thereof.

7. The method of any one of clauses 1 to 6, wherein the data for the plurality of pre-analytical variables is supplemented with data for tumor cellularity in the sample, tumor nuclei content in the sample, tissue surface area of the sample, tissue matrix in the sample, or any combination thereof.

8. The method of any one of clauses 1 to 7, wherein the multivariable model comprises a machine learning model.

9. The method of clause 8, wherein the machine learning model comprises a supervised learning model.

10. The method of clause 8, wherein the machine learning model comprises an unsupervised learning model.

11. The method of any one of clauses 1 to 10, wherein the multivariable model comprises a logistic regression model, a multiple linear regression model, a random forest model, a neural network model, or a deep learning model.

12. The method of any one of clauses 1 to 11, wherein the prediction of the likelihood of success comprises a binary value, a percentage, or a score.

13. The method of any one of clauses 1 to 11, further comprising:
comparing the predicted likelihood of success to a predefined threshold; and
based on a determination that the predicted likelihood of success is greater than or equal to a predefined threshold, outputting an indication that the sample from the subject is suitable for providing a genomic profile of the subject.

14. The method of any one of clauses 1 to 11, further comprising:
comparing the predicted likelihood of success to a predefined threshold; and
based on a determination that the predicted likelihood of success is less than the predefined threshold, outputting a recommendation for collecting a new sample instead of submitting the sample for genomic profiling.

15. The method of clause 14, further comprising outputting a recommendation for sample type or sample collection site for the new sample.

16. The method of clause 14 or clause 15, further comprising outputting a recommendation for an alternative nucleic acid sequencing-based test method to perform.

17. The method of any one of clauses 2 to 16, wherein the predefined threshold varies depending on sample type.

18. The method of any one of clauses 1 to 17, wherein the data for the plurality of pre-analytical variables is input by a user via a graphical user interface (GUI) on a display device.

19. The method of any one of clauses 1 to 18, wherein the prediction of the likelihood of success for performing genomic profiling of the sample is reported via a graphical user interface (GUI) on a display device.

20. The method of clause 18 or clause 19, wherein the graphical user interface (GUI) is displayed in a web browser.

21. The method of any one of clauses 1 to 20, wherein the data received for the plurality of pre-analytical variables includes data for sample type.

22. The method of clause 21, wherein the remaining pre-analytical variables of the plurality of pre-analytical variables are selected based on the sample type.

23. The method of clause 21 or clause 22, wherein the multivariable model is selected based on the sample type.

24. The method of any one of clauses 1 to 23, wherein the sample comprises a tissue biopsy sample, a liquid biopsy sample, or a normal control.

25. The method of clause 24, wherein the sample is a tissue biopsy sample and comprises bone marrow.

26. The method of clause 24, wherein the sample is a liquid biopsy sample and comprises blood, plasma, cerebrospinal fluid, sputum, stool, urine, or saliva.

27. The method of clause 24, wherein the sample is a liquid biopsy sample and comprises circulating tumor cells (CTCs).

28. The method of clause 24, wherein the sample is a liquid biopsy sample and comprises cell-free DNA (cfDNA), circulating tumor DNA (ctDNA), or any combination thereof.

29. The method of any one of clauses 13 to 28, wherein, if the predicted likelihood of success is greater than or equal to the predefined threshold, the genomic profiling is performed and used to diagnose or confirm a diagnosis of disease in the subject.

30. The method of clause 29, wherein the genomic profiling is also used to determine eligibility for therapy based on a biomarker status.

31. The method of clause 29 or clause 30, wherein the disease is cancer.

32. The method of clause 31, further comprising selecting an anti-cancer therapy to administer to the subject based on the results of the genomic profiling.

33. The method of clause 31 or clause 32, further comprising determining an effective amount of an anti-cancer therapy to administer to the subject based on the results of the genomic profiling.

34. The method of clause 33, further comprising administering the anti-cancer therapy to the subject based on the results of the genomic profiling.

35. The method of any one of clauses 32 to 34, wherein the anti-cancer therapy comprises chemotherapy, radiation therapy, immunotherapy, a targeted therapy, or surgery.

36. The method of any one of clauses 31 to 35, wherein the cancer is a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

37. The method of any one of clauses 29 to 36, wherein the genomic profiling for the subject comprises obtaining results from a comprehensive genomic profiling (CGP) test, a gene expression profiling test, a cancer hotspot panel test, a DNA methylation test, a DNA fragmentation test, an RNA fragmentation test, or any combination thereof.

38. The method of clause 37, wherein the genomic profiling for the subject further comprises obtaining results from a nucleic acid sequencing-based test.

39. The method of clause 37 or clause 38, further comprising selecting an anti-cancer agent, administering an anti-cancer agent, or applying an anti-cancer treatment to the subject based on the genomic profiling results.

40. A system comprising:
one or more processors; and
a memory communicatively coupled to the one or more processors and configured to store instructions that, when executed by the one or more processors, cause the system to:
   receive the data for a plurality of pre-analytical variables associated with a sample derived from a subject;
   apply the received data to a multivariable model trained to predict outcomes for genomic profiling assays;
   generate a prediction of the likelihood of success for performing genomic profiling of the sample based on the applied multivariable model; and
   report the prediction of the likelihood of success for performing genomic profiling of the sample.

41. The system of clause 40, wherein the instructions further cause the system to compare the predicted likelihood of success to a predefined threshold; and based on a determination that the predicted likelihood of success is less than the predefined threshold, output a recommendation for collecting a new sample instead of submitting the sample for genomic profiling.

42. The system of clause 40 or clause 41, wherein the data for the plurality of pre-analytical variables is input by a user via a graphical user interface (GUI) on a display device.

43. The system of any one of clauses 40 to 42, wherein the prediction of the likelihood of success for performing genomic profiling of the sample is reported via a graphical user interface (GUI) on a display device.

44. The system of clause 42 or clause 43, wherein the graphical user interface (GUI) is displayed in a web browser.

45. A non-transitory computer-readable storage medium storing one or more programs, the one or more programs comprising instructions, which when executed by one or more processors of a system, cause the system to:
receive the data for a plurality of pre-analytical variables associated with a sample derived from a subject;
apply the received data to a multivariable model trained to predict outcomes for genomic profiling assays;
generate a prediction of the likelihood of success for performing genomic profiling of the sample based on the applied multivariable model; and
report the prediction of the likelihood of success for performing genomic profiling of the sample.

46. The non-transitory computer-readable storage medium of clause 45, wherein the instructions further cause the system to compare the predicted likelihood of success to a predefined threshold; and based on a determination that the predicted likelihood of success is less than the predefined threshold, output a recommendation for collecting a new sample instead of submitting the sample for genomic profiling.

It should be understood from the foregoing that, while particular implementations of the disclosed methods and systems have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

1. A method for predicting a likelihood of success for performing genomic profiling of a sample derived from a subject, comprising:
receiving, using one or more processors, data for a plurality of pre-analytical variables associated with the sample;
applying, using the one or more processors, the received data to a multivariable model trained to predict outcomes for genomic profiling assays;
generating, using the one or more processors, a prediction of the likelihood of success for performing genomic profiling of the sample based on the applied multivariable model;
based on the prediction of the likelihood of success being equal to or greater than a predefined threshold, providing a plurality of nucleic acid molecules obtained from the sample from the subject;
ligating one or more adapters onto one or more nucleic acid molecules from the plurality of nucleic acid molecules;
amplifying the one or more ligated nucleic acid molecules from the plurality of nucleic acid molecules;
capturing amplified nucleic acid molecules from the amplified nucleic acid molecules;
sequencing, by a sequencer, the captured nucleic acid molecules to obtain a plurality of sequence reads that represent the captured nucleic acid molecules and that overlap one or more gene loci within one or more subgenomic intervals in the sample; and
generating, by the one or more processors, a genomic profile including sequence read analysis data based on the sequence reads for the sample.

2. (canceled)

3. The method of claim 1, further comprising:
training, by the one or more processors, the multivariable model using training data.

4. The method of claim 3, wherein the training data comprises data derived from univariate analyses of clinical study data for samples collected from subjects representing a range of subject age, subject sex, diagnosis, stage of disease, sample type, sample collection site, sample collection method, sample preparation method, sample preservation method, sample age, sample transportation method, or any combination thereof.

5. The method of claim 4, wherein the training data further comprises data for ECOG status, subject treatment status, tumor cellularity in the sample, tumor nuclei content in the sample, tissue surface area of the sample, tissue matrix in the sample, prior genomic profiling assay results, or any combination thereof.

6. The method of claim 1, wherein the plurality of pre-analytical variables comprises subject age, subject sex, diagnosis, stage of disease, sample type, sample collection site, sample collection method, sample preparation method, sample preservation method, sample age, sample transportation method, or any combination thereof.

7. The method of claim 1, wherein the data for the plurality of pre-analytical variables is supplemented with data for tumor cellularity in the sample, tumor nuclei content in the sample, tissue surface area of the sample, tissue matrix in the sample, or any combination thereof.

8. The method of claim 1, wherein the multivariable model comprises a machine learning model.

9. (canceled)

10. (canceled)

11. The method of claim 1, wherein the multivariable model comprises a logistic regression model, a multiple linear regression model, a random forest model, a neural network model, or a deep learning model.

12. The method of claim 1, wherein the prediction of the likelihood of success comprises a binary value, a percentage, or a score.

13. The method of claim 1, further comprising:
comparing the predicted likelihood of success to a predefined threshold; and
based on a determination that the predicted likelihood of success is greater than or equal to a predefined threshold, outputting an indication that the sample from the subject is suitable for providing a genomic profile of the subject.

14. The method of claim 1, further comprising:
comparing the predicted likelihood of success to a predefined threshold; and
based on a determination that the predicted likelihood of success is less than the predefined threshold, outputting a recommendation for collecting a new sample instead of submitting the sample for genomic profiling.

15. (canceled)

16. (canceled)

17. The method of claim 2, wherein the predefined threshold varies depending on sample type.

18. (canceled)

19. (canceled)

20. (canceled)

21. The method of claim 1, wherein the data received for the plurality of pre-analytical variables includes data for sample type.

22. The method of claim 21, wherein the remaining pre-analytical variables of the plurality of pre-analytical variables are selected based on the sample type and/or wherein the multivariable model is selected based on the sample type.

23. (canceled)

24. (canceled)

25. The method of claim 1, wherein the sample is a tissue biopsy sample and comprises bone marrow.

26. (canceled)

27. The method of claim 1, wherein the sample is a liquid biopsy sample and comprises circulating tumor cells (CTCs), cell-free DNA (cfDNA), circulating tumor DNA (ctDNA), or any combination thereof.

28. (canceled)

29. The method of claim 13, wherein, if the predicted likelihood of success is greater than or equal to the predefined threshold, the genomic profiling is performed and used to diagnose or confirm a diagnosis of cancer in the subject.

30. The method of claim 29, wherein the genomic profiling is also used to determine eligibility for anti-cancer therapy based on a biomarker status.

31. (canceled)

32. The method of claim 29, further comprising selecting an anti-cancer therapy to administer to the subject, determining an effective amount of an anti-cancer therapy to administer to the subject, and/or administering the anti-cancer therapy to the subject based on the results of the genomic profiling.

33. (canceled)

34. (canceled)

35. (canceled)

36. (canceled)

37. (canceled)

38. (canceled)

39. (canceled)

40. A system comprising:
one or more processors; and
a memory communicatively coupled to the one or more processors and configured to store instructions that, when executed by the one or more processors, cause the system to:
receive the data for a plurality of pre-analytical variables associated with a sample derived from a subject;
apply the received data to a multivariable model trained to predict outcomes for genomic profiling assays;
generate a prediction of the likelihood of success for performing genomic profiling of the sample based on the applied multivariable model; and
report the prediction of the likelihood of success for performing genomic profiling of the sample.

41. (canceled)

42. (canceled)

43. (canceled)

44. (canceled)

45. A non-transitory computer-readable storage medium storing one or more programs, the one or more programs comprising instructions, which when executed by one or more processors of a system, cause the system to:
receive the data for a plurality of pre-analytical variables associated with a sample derived from a subject;
apply the received data to a multivariable model trained to predict outcomes for genomic profiling assays;
generate a prediction of the likelihood of success for performing genomic profiling of the sample based on the applied multivariable model; and
report the prediction of the likelihood of success for performing genomic profiling of the sample.

46. (canceled)
